(19) United States
(12) Patent Application Publication
(10) Pub. No.: US 2025/0084163 A1
(43) Pub. Date: Mar. 13, 2025

(54) Antigen Binding Proteins That Bind ROR1

(71) Applicant: Sorrento Therapeutics, Inc., San Diego, CA (US)

(72) Inventors: Heyue Zhou, San Diego, CA (US); Xia Cao, San Diego, CA (US); Lucy Lu, San Diego, CA (US)

(73) Assignee: Sorrento Therapeutics, Inc., San Diego, CA (US)

(21) Appl. No.: 18/554,379

(22) PCT Filed: Apr. 8, 2022
(86) PCT No.: PCT/US2022/024030

§ 371 Date: Oct. 6, 2023

Related U.S. Application Data
(60) Provisional application No. 63/173,150, filed on Apr. 9, 2021.

(51) Int. Cl. C07K 16/28

(52) U.S. Cl. CPC C07K 16/2803; C07K 2317/21; C07K 2317/33; C07K 2317/92

(57) ABSTRACT

The present disclosure provides ROR1 binding proteins, particularly anti-ROR1 antibodies, or antigen-binding portions thereof, that specifically bind ROR1 and uses thereof. Various aspects of the anti-ROR1 antibodies relate to antibody fragments, single-chain antibodies, pharmaceutical compositions, nucleic acids, recombinant expression vectors, host cells, and methods for preparing and using such anti-ROR1 antibodies. Methods for using the anti-ROR1 antibodies include in vitro and in vivo methods for binding ROR1, detecting ROR1 and treating diseases associated with ROR1 expression.

This application claims the benefit of priority under 35 U.S.C. § 119 to U.S. provisional application No. 63/173,150, filed Apr. 9, 2021, the entire contents of which are incorporated by reference in its entirety.

### SEQUENCE LISTING

The present application is filed with a Sequence Listing in electronic format. The Sequence Listing is provided as a file entitled “2022-04-01_01223-0097-00PCT_Seq_List_ST25.txt” created on Apr. 1, 2022, which is 6,160 bytes in size. The information in the electronic format of the sequence listing is incorporated herein by reference in its entirety.

Throughout this application various publications, patents, and/or patent applications are referenced. The disclosures of the publications, patents and/or patent applications are hereby incorporated by reference in their entireties into this application in order to more fully describe the state of the art to which this disclosure pertains.

### TECHNICAL FIELD

The present disclosure provides antigen binding proteins that bind specifically to ROR1 and nucleic acids that encode the antigen binding proteins, vectors comprising the nucleic acids, host cells harboring the vectors, and method of use thereof.

### BACKGROUND

Receptor tyrosine kinase-like orphan receptors (ROR) belong to a highly conserved family of receptor tyrosine kinases, which consists of two family members, ROR1 and ROR2, which are type-I transmembrane receptor tyrosine kinases. Members of the ROR family are type-I transmembrane proteins containing three distinct extracellular domains, an Ig, a Kringle and a Frizzled domain, followed a transmembrane spanning region, and an intracellular portion. Within the intracellular portion, ROR 1 possesses a tyrosine kinase domain, two serine/threonine-rich domains and a proline-rich domain.

Receptor tyrosine kinases (RTKs) play a key role in oncogenic transformation, growth and metastases. RTKs regulate cell differentiation, proliferation, migration, angiogenesis, and survival.

The cellular function of this family is to regulate cell migration, planar cell polarity (PCP) and apical-basal cell polarity, and axon outgrowth in developmental processes, including skeletal and neuronal development. Wnt5a, a glycoprotein critical in carcinogenesis, has been identified as regulating these functions by binding and activating ROR1 and ROR2 (Nishita et al., 2010, Trends Cell Biol., 20 (6), 346-54). Wnt5a binding to ROR2 and its co-receptor, Frizzled domain, can activate the INK pathway and filamin A to regulate cell migration and invasion, cause Racl and Rho A to regulate cell polarity, and induce Src family members to modulate the expression of matrix metalloproteases, such as MMP 1, 2, 13, and inhibit the canonical Wnt pathways.

ROR1 promotes cell proliferation through NF-kB when co-expressed with Wnt5a (Fukuda et al., 2008, Proc. Natl. Acad. Sci. U.S.A., 105 (8): 3047-52). Functional data suggest that ROR1 may function in non-canonical WNT-signaling to promote the survival of malignant cells.

Receptor tyrosine kinase orphan receptors-1 and -2 (ROR1 and ROR2) have been described as being specifically associated with particular cancers (Rebagay et al., 2012, Front Oncol., 2 (34)), while being largely absent in expression on healthy tissue with few exceptions (Balakrishnan et al., 2017, Clin. Cancer Res., 23 (12), 3061-3071). Due to the very tumor-selective expression of the ROR family members, they represent relevant targets for targeted cancer therapies.

ROR1 is aberrantly expressed in B-cell chronic lymphocytic leukemia (CLL) and mantle cell lymphoma (MCL). Receptor tyrosine kinase orphan receptors-1 (ROR1) exhibits nearly 100% association with chronic lymphocytic leukemia (CLL) (Cui et al., 2016, Blood, 128 (25), 2931) and it is also expressed in certain solid tumors, like that of lung and breast (Balakrishnan et al., 2017, Clin. Cancer Res., 23 (12), 3061-3071). Additionally, ROR1 has been established as a marker for some acute lymphoblastic leukemias (ALL), mantle cell lymphomas, and some other blood malignancies. ROR1 is critically involved in progression of a number of solid tumors, such as in neuroblastoma, sarcoma, renal cell carcinoma, breast cancer, lung cancer, colon cancer, head and neck cancer, melanoma, and other cancers. ROR1 has been shown to inhibit apoptosis, potentiate EGFR signaling, induce epithelial-mesenchymal transition (EMT), and contribute to caveolae formation.

Importantly, ROR1 is mainly detectable in embryonic tissue and generally absent in adult tissue, making the protein an ideal drug target for cancer therapy. As such, ROR1 has previously been recognized as a target for the development of ROR1 specific antibodies. However, due to the high homology of ROR1 between different mammalian species, which is 100% conserved on the amino acid level between humans and cynomolgus monkeys, 96.7% homologous between human and mouse, and 96.3% homologous between human and rabbit, it has been difficult to raise high affinity antibodies against this target by standard technologies, like animal immunizations.

Due to the low number of available ROR1 specific monoclonal antibodies, there is a need in the art for better anti-ROR1 antibodies that have higher affinity or other functional properties not possessed by the known antibody clones.

Thus, ROR1 is an attractive antigen for targeting with antibodies. The present disclosure provides ROR1 binding proteins, particularly anti-ROR1 antibodies or antigen-binding portions thereof, that specifically bind ROR1, and uses thereof.

### SUMMARY

In one aspect, provided herein is an anti-ROR1 antigen-binding protein or fully human anti-ROR1 antibody, or an antigen-binding fragment thereof, comprising a heavy chain variable region and a light chain variable region, wherein the heavy chain variable region comprises a heavy chain complementarity determining region 1 (CDR1) a heavy chain CDR2 and a heavy chain CDR3, and the light chain variable region comprises a light chain CDR1, a light chain CDR2, and a light chain CDR3; and (a) the heavy chain CDR1 has the amino acid sequence of SEQ ID NO:12, the heavy chain CDR2 has the amino acid sequence of SEQ ID NO: 13, the heavy chain CDR3 has the amino acid sequence of SEQ ID NO: 14, the light chain CDR 1 has the amino acid sequence of SEQ ID NO: 15, the light chain CDR2 has the amino acid sequence of SEQ ID NO:16, and the light chain CDR3 has the amino acid sequence of SEQ ID NO: 17; (b) the heavy chain CDR1 has the amino acid sequence of SEQ ID NO:22, the heavy chain CDR2 has the amino acid sequence of SEQ ID NO:23, the heavy chain CDR3 has the amino acid sequence of SEQ ID NO:24, the light chain CDR 1 has the amino acid sequence of SEQ ID NO: 25, the light chain CDR2 has the amino acid sequence of SEQ ID NO:26, and the light chain CDR3 has the amino acid sequence of SEQ ID NO:27; (c) the heavy chain CDR1 has the amino acid sequence of SEQ ID NO:32, the heavy chain CDR2 has the amino acid sequence of SEQ ID NO: 33, the heavy chain CDR3 has the amino acid sequence of SEQ ID NO:34, the light chain CDR 1 has the amino acid sequence of SEQ ID NO:35, the light chain CDR2 has the amino acid sequence of SEQ ID NO:36, and the light chain CDR3 has the amino acid sequence of SEQ ID NO: 37; (d) the heavy chain CDR1 has the amino acid sequence of SEQ ID NO:42, the heavy chain CDR2 has the amino acid sequence of SEQ ID NO:43, the heavy chain CDR3 has the amino acid sequence of SEQ ID NO:44, the light chain CDR1 has the amino acid sequence of SEQ ID NO:45, the light chain CDR2 has the amino acid sequence of SEQ ID NO:46, and the light chain CDR3 has the amino acid sequence of SEQ ID NO:47; (e) the heavy chain CDR1 has the amino acid sequence of SEQ ID NO:42, the heavy chain CDR2 has the amino acid sequence of SEQ ID NO:43, the heavy chain CDR3 has the amino acid sequence of SEQ ID NO:44, the light chain CDR1 has the amino acid sequence of SEQ ID NO:55, the light chain CDR2 has the amino acid sequence of SEQ ID NO:56, and the light chain CDR3 has the amino acid sequence of SEQ ID NO:57; In embodiments, the heavy chain variable region has at least 95% sequence identity to the amino acid sequence of SEQ ID NO: 10, 20,30 or 40, and the light chain variable region has at least 95% sequence identity to the amino acid sequence of SEQ ID NO:11, 21, 31, 41 or 51.

In an aspect, provided herein is an antigen-binding protein or fully human anti-ROR1 antibody, or an antigen-binding fragment thereof, comprising a heavy chain variable region and a light chain variable region, the heavy chain variable region having at least 95% sequence identity to the amino acid sequence of SEQ ID NO:10, 20, 30 or 40, and the light chain variable region having at least 95% sequence identity to the amino acid sequence of SEQ ID NO:11, 21, 31, 41 or 51.

In an aspect, provided herein is an antigen-binding protein or fully human anti-ROR1 antibody, or an antigen-binding fragment thereof, comprising a heavy chain variable region and a light chain variable region, wherein the heavy chain variable region and the light chain variable region comprise the amino acid sequences of SEQ ID NOS: 10 and 11, respectively (e.g., herein called RO6D8-s10), SEQ ID NOS: 20 and 21, respectively (e.g., herein called RO6D8-jlv1011), SEQ ID NOS: 30 and 31, respectively (e.g., herein called RO6D8-011), SEQ ID NOS: 40 and 41, respectively (e.g., herein called RO6A-a7gm), or SEQ ID NOS: 40 and 51, respectively (e.g., herein called RO6A-a8gm).

In embodiments, the antigen binding fragment includes a Fab fragment. In embodiments, the antigen binding fragment includes a single chain antibody, wherein the heavy chain variable domain and the light chain variable domain are joined together with a peptide linker. In embodiments, any one of the disclosed antigen-binding protein, antibody or antigen-binding fragment thereof, include an IgG antibody, which is IgG1, IgG2, IgG3 or IgG4 class antibody. In embodiments, any one of the disclosed antigen-binding protein, antibody or antigen-binding fragment thereof, include IgG1 or IgG4 antibody. In embodiments, any one of the disclosed antigen-binding protein, antibody or antigen-binding fragment thereof, include IgG1 antibody. In embodiments, any one of the disclosed antigen-binding protein, antibody or antigen-binding fragment thereof, binds human ROR1 protein with a K_{D} of 10⁻⁷ M or less.

In an aspect, provided herein is a pharmaceutical composition, including any one of the disclosed antigen-binding protein, antibody or antigen-binding fragment and a pharmaceutically acceptable excipient.

In an aspect, provided herein is a kit including any one of the disclosed antigen-binding protein, antibody or antigen-binding fragments and a pharmaceutically acceptable excipient.

In an aspect, provided herein is a nucleic acid that encodes the heavy chain variable region of any one of the disclosed antigen-binding protein, antibody or antigen-binding fragment.

In an aspect, provided herein is a nucleic acid that encodes the light chain variable region of any one of the disclosed antigen-binding protein, antibody or antigen-binding fragment.

In an aspect, provided herein is a nucleic acid that encodes (i) the heavy chain variable region of any one of the disclosed antigen-binding protein, antibody or antigen-binding fragment, and (ii) the light chain variable region of any one of the disclosed antigen-binding protein, antibody or antigen-binding fragment.

In an aspect, provided herein is a vector including any one of the disclosed nucleic acids.

In an aspect, provided herein is a host cell harboring any of the disclosed vectors. In embodiments, the disclosed vector includes an expression vector, and the host cell expresses the heavy chain variable region. In embodiments, the disclosed vector includes an expression vector, and the host cell expresses the light chain variable region.

In an aspect, provided herein is a host cell harboring a first vector and a second vector. In embodiments the first vector comprises a first expression vector, the second vector comprises a second expression vector, and the host cell expresses the heavy and the light chain variable regions.

In an aspect, provided herein is a method for preparing a heavy chain variable region of an antigen-binding protein, antibody or antigen-binding fragment, the method comprising: culturing a population of the host cell under conditions suitable for expressing the heavy chain variable region of the antigen-binding protein, antibody or antigen-binding fragment. In embodiments, the method further includes recovering from the host cells the expressed heavy chain variable region of the antigen-binding protein, antibody or antigen-binding fragment.

In an aspect, provided herein is a method for preparing a light chain variable region of an antigen-binding protein, antibody or antigen-binding fragment, the method comprising: culturing a population of the host cell under conditions suitable for expressing the light chain variable region of the antigen-binding protein, antibody or antigen-binding fragment. In embodiments, the method further includes recovering from the host cells the expressed light chain variable region of the antigen-binding protein, antibody or antigen-binding fragment.

In an aspect, provided herein is a method for preparing (i) a heavy chain variable region of an antigen-binding protein, antibody or antigen-binding fragment, and (ii) a light chain variable region of an antigen-binding protein, antibody or antigen-binding fragment, the method comprising: culturing a population of the host cell under conditions suitable for expressing (i) the heavy chain variable region of the antigen-binding protein, antibody or antigen-binding fragment, and (ii) the light chain variable region of the antigen-binding protein, antibody or antigen-binding fragment. In embodiments, the method further includes recovering from the host cells (i) the expressed heavy chain variable region of the antigen-binding protein, antibody or antigen-binding fragment, and (ii) the expressed light chain variable region of the antigen-binding protein, antibody or antigen-binding fragment.

In an aspect, provided herein is a method for inhibiting growth or proliferation of ROR1-expressing cells, comprising: contacting a population of effector cells with a population of target cells which express ROR1, in the presence of the human anti-ROR1 antibody of any one of the disclosed antigen-binding protein, antibody or antigen-binding fragment, under conditions that are suitable for inhibiting growth or proliferation of the ROR1-expressing cells. In embodiments, the population of effector cells comprises PBMCs or NK cells. In embodiments, the population of target cells comprise ROR1 expressing human cancer cells or transgenic cells expressing ROR1. In embodiments, the ratio of the effector-to-target cells is 1:1, 2:1, 3:1, 4:1 or 5:1. In embodiments, the ratio of the effector-to-target cells is 5-10:1, 10-20:1, or 20-30:1.

In an aspect, provided herein is a method for killing ROR1-expressing cells, comprising: contacting a population of effector cells with a population of target cells which express ROR1 in the presence of the human anti-ROR1 antibody of any one of the disclosed antigen-binding protein, antibody or antigen-binding fragment, under conditions that are suitable for inhibiting growth or proliferation of the ROR1-expressing cells. In embodiments, the population of effector cells comprises PBMCs or NK cells. In embodiments, the population of target cells comprise ROR1 expressing human cancer cells or transgenic cells expressing ROR1. In embodiments, the ratio of the effector-to-target cells is 1:1, 2:1, 3:1, 4:1 or 5:1. In embodiments, the ratio of the effector-to-target cells is 5-10:1, 10-20:1, or 20-30:1.

In an aspect, provided herein is a method for treating a subject having a disease associated with ROR1 expression, the method comprising: administering to the subject an effective amount of a therapeutic composition comprising the antigen-binding protein, antibody or antigen-binding fragment of any one of the disclosed antigen-binding protein, antibody or antigen-binding fragment. In embodiments, the disease associated with ROR1 expression is cancer. In embodiments, the cancer is chronic lymphocytic leukemia (CLL), breast cancer, lung cancer, gastric cancer, melanoma, colon cancer, renal cell carcinoma, or lymphomas. In embodiments, the cancer is chronic lymphocytic leukemia (CLL), hairy cell leukemia (HCL), mantle cell lymphoma (MCL), diffuse large B-cell lymphoma (DLBCL), marginal zone lymphoma (MZL), follicular lymphoma (FL), chronic myeloid leukemia (CML), acute myeloid lymphoma (AML), myeloma, T-cell leukemia (TCL), Burkitt's lymphoma, multiple myeloma (MM), small lymphocytic lymphoma (SLL), non-Hodgkin lymphoma (NHL) that has undergone Richter's transformation, non-small cell lung cancer (NSCLC), hepatocellular carcinoma, pancreatic cancer, osteosarcoma, head and neck cancer, ovarian cancer, breast cancer, or triple negative breast cancer (TNBC). lymphoma, small lymphocytic lymphoma, marginal cell B-cell lymphoma, renal cell carcinoma, colon cancer, colorectal cancer, epithelial squamous cell cancer, melanoma, myeloma, stomach cancer, brain cancer, lung cancer, cervical cancer, liver cancer, bladder cancer, prostate cancer, testicular cancer, or thyroid cancer. In embodiments, the cancer is a metastatic cancer, refractory cancer, or recurrent cancer.

### DESCRIPTION OF THE DRAWINGS

FIG. 1A shows an SPR sensorgram of binding kinetics of RO6D8wt antibody.

FIG. 1B shows an SPR sensorgram of binding kinetics of RO6D8-s10 antibody.

FIG. 1C shows an SPR sensorgram of binding kinetics of RO6D8-jlv1011 antibody.

FIG. 1D shows an SPR sensorgram of binding kinetics of RO6D8-011 antibody.

FIG. 1E shows a table that summarizes binding kinetics of antibodies RO6D8wt, RO6D8-s10, RO6D8-jlv1011, and RO6D8-011 with ROR1 antigen, obtained from SPR data of FIGS. 1A-1D.

FIG. 2A is a bar graph showing the results of an ELISA assay for various anti-ROR1 antibodies cross-reactivity with human and mouse ROR1 protein.

FIG. 2B shows a graph of mouse cross-reactivity, as a function of antibody concentration, for RO6D8-s10 and RO6D8-jlv1011.

FIG. 3A shows graphs of cell binding assay of anti-ROR1 antibody RO6D8wt, binding to ROR1-expressing cells A549 and ROR1 negative cells Jurkat.

FIG. 3B shows graphs of cell binding assay of anti-ROR1 antibody RO6D8-s10, binding to ROR1-expressing cells A549 and ROR1 negative cells Jurkat.

FIG. 3C shows graphs of cell binding assay of anti-ROR1 antibody RO6D8-jlv1011, binding to ROR1-expressing cells A549 and ROR1 negative cells Jurkat.

FIGS. 4A-E show graphs of cell binding assays of anti-ROR1 antibodies RO6D8-s10, RO6D8-Jlv1011, RO6A-a7gm, and RO6A-a8gm, binding to ROR 1-expressing cells A549 (FIG. 4A), RAJI (FIG. 4B), and MCF7 (FIG. 4C) and ROR1 negative cells A549 ROR1-KO (FIG. 4D) and LS174T (FIG. 4E).

### DESCRIPTION

### Definitions:

Unless defined otherwise, technical and scientific terms used herein have meanings that are commonly understood by those of ordinary skill in the art unless defined otherwise. Generally, terminologies pertaining to techniques of cell and tissue culture, molecular biology, immunology, microbiology, genetics, transgenic cell production, protein chemistry and nucleic acid chemistry and hybridization described herein are well known and commonly used in the art. The methods and techniques provided herein are generally performed according to conventional procedures well known in the art and as described in various general and more specific references that are cited and discussed herein unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992). A number of basic texts describe standard antibody production processes, including, Borrebaeck (ed) Antibody Engineering, 2nd Edition Freeman and Company, NY, 1995; McCafferty et al. Antibody Engineering, A Practical Approach IRL at Oxford Press, Oxford, England, 1996; and Paul (1995) Antibody Engineering Protocols Humana Press, Towata, N.J., 1995; Paul (ed.), Fundamental Immunology, Raven Press, N.Y, 1993; Coligan (1991) Current Protocols in Immunology Wiley/Greene, NY; Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; Coding Monoclonal Antibodies: Principles and Practice (2nd ed.) Academic Press, New York, N.Y., 1986, and Kohler and Milstein Nature 256:495-497, 1975. All of the references cited herein are incorporated herein by reference in their entireties. Enzymatic reactions and enrichment/purification techniques are also well known and are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are well known and commonly used in the art. Standard techniques can be used for chemical syntheses, chemical analyses, pharmaceutical preparation, formulation, and delivery, and treatment of patients.

The headings provided herein are not limitations of the various aspects of the disclosure, which aspects can be understood by reference to the specification as a whole.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative (e.g., “or”) herein is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of” and/or “consisting essentially of” are also provided.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within one or more than one standard deviation per the practice in the art. Alternatively, “about” or “comprising essentially of” can mean a range of up to 10% (i.e., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “comprising essentially of” should be assumed to be within an acceptable error range for that particular value or composition.

The terms “peptide”, “polypeptide” and “protein” and other related terms used herein are used interchangeably and refer to a polymer of amino acids and are not limited to any particular length. Polypeptides may comprise natural and non-natural amino acids. Polypeptides include recombinant or chemically-synthesized forms. Polypeptides also include precursor molecules and mature molecule. Precursor molecules include those that have not yet been subjected to cleavage, for example cleavage by a secretory signal peptide or by non-enzymatic cleavage at certain amino acid residue. Polypeptides in include mature molecules that have undergone cleavage. These terms encompass native and artificial proteins, protein fragments and polypeptide analogs (such as muteins, variants, chimeric proteins and fusion proteins) of a protein sequence as well as post-translationally, or otherwise covalently or non-covalently, modified proteins. Polypeptides comprising amino acid sequences of binding proteins that bind ROR1 (e.g., anti-ROR1 antibodies or antigen-binding portions thereof) prepared using recombinant procedures are described herein.

The terms “nucleic acid”, “polynucleotide” and “oligonucleotide” and other related terms used herein are used interchangeably and refer to polymers of nucleotides and are not limited to any particular length. Nucleic acids include recombinant and chemically-synthesized forms. Nucleic acids include DNA molecules (cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. Nucleic acid molecule can be single-stranded or double-stranded. In embodiments, the nucleic acid molecules of the disclosure comprise a contiguous open reading frame encoding an antibody, or a fragment or scFv, derivative, mutein, or variant thereof. In embodiments, nucleic acids comprise a one type of polynucleotides or a mixture of two or more different types of polynucleotides. Nucleic acids encoding anti-ROR1 antibodies or antigen-binding portions thereof, are described herein.

The term “recover” or “recovery” or “recovering”, and other related terms, refers to obtaining a protein (e.g., an antibody or an antigen binding portion thereof), from host cell culture medium or from host cell lysate or from the host cell membrane. In embodiments, the protein is expressed by the host cell as a recombinant protein fused to a secretion signal peptide sequence which mediates secretion of the expressed protein. The secreted protein can be recovered from the host cell medium. In embodiments, the protein is expressed by the host cell as a recombinant protein that lacks a secretion signal peptide sequence which can be recovered from the host cell lysate. In embodiments, the protein is expressed by the host cell as a membrane-bound protein which can be recovered using a detergent to release the expressed protein from the host cell membrane. In embodiments, irrespective of the method used to recover the protein, the protein can be subjected to procedures that remove cellular debris from the recovered protein. For example, the recovered protein can be subjected to chromatography, gel electrophoresis and/or dialysis. In embodiments, the chromatography comprises any one or any combination or two or more procedures including affinity chromatography, hydroxyapatite chromatography, ion-exchange chromatography, reverse phase chromatography and/or chromatography on silica. In embodiments, affinity chromatography comprises protein A or G (cell wall components from Staphylococcus aureus).

The term “isolated” refers to a protein (e.g., an antibody or an antigen binding portion thereof) or polynucleotide that is substantially free of other cellular material. A protein may be rendered substantially free of naturally associated components (or components associated with a cellular expression system or chemical synthesis methods used to produce the antibody) by isolation, using protein purification techniques well known in the art. The term isolated also refers in some embodiments to protein or polynucleotides that are substantially free of other molecules of the same species, for example other protein or polynucleotides having different amino acid or nucleotide sequences, respectively. The purity of homogeneity of the desired molecule can be assayed using techniques well known in the art, including low resolution methods such as gel electrophoresis and high resolution methods such as HPLC or mass spectrophotometry. In embodiments, any of the anti-ROR1 antibodies or antigen binding protein thereof are isolated.

An “antigen binding protein” and related terms used herein refers to a protein comprising a portion that binds to an antigen and, optionally, a scaffold or framework portion that allows the antigen binding portion to adopt a conformation that promotes binding of the antigen binding protein to the antigen. Examples of antigen binding proteins include antibodies, antibody fragments (e.g., an antigen binding portion of an antibody), antibody derivatives, and antibody analogs. The antigen binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding protein as well as wholly synthetic scaffolds comprising, for example, a biocompatible polymer. See, for example, Korndorfer et al., 2003, Proteins: Structure, Function, and Bioinformatics, Volume 53, Issue 1:121-129; Roque et al., 2004, Biotechnol. Prog. 20:639-654. In addition, peptide antibody mimetics (“PAMs”) can be used, as well as scaffolds based on antibody mimetics utilizing fibronection components as a scaffold. Antigen binding proteins that bind ROR1 are described herein.

An antigen binding protein can have, for example, the structure of an immunoglobulin. In embodiments, an “immunoglobulin” refers to a tetrameric molecule composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa or lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site such that an intact immunoglobulin has two antigen binding sites. In embodiments, an antigen binding protein can be a synthetic molecule having a structure that differs from a tetrameric immunoglobulin molecule but still binds a target antigen or binds two or more target antigens. For example, a synthetic antigen binding protein can comprise antibody fragments, 1-6 or more polypeptide chains, asymmetrical assemblies of polypeptides, or other synthetic molecules. Antigen binding proteins having immunoglobulin-like properties that bind specifically to ROR1 are described herein.

The variable regions of immunoglobulin chains exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3 and FR4.

One or more CDRs may be incorporated into a molecule either covalently or noncovalently to make it an antigen binding protein. An antigen binding protein may incorporate the CDR(s) as part of a larger polypeptide chain, may covalently link the CDR(s) to another polypeptide chain, or may incorporate the CDR(s) noncovalently. The CDRs permit the antigen binding protein to specifically bind to a particular antigen of interest.

The assignment of amino acids to each domain is in accordance with the definitions of Kabat et al. in Sequences of Proteins of Immunological Interest, 5^{th} Ed., US Dept. of Health and Human Services, PHS, NIH, NIH Publication no. 91-3242, 1991. Other numbering systems for the amino acids in immunoglobulin chains include IMGT.RTM. (international ImMunoGeneTics information system; Lefranc et al, Dev. Comp. Immunol. 29:185-203; 2005) and Aho (Honegger and Pluckthun, J. Mol. Biol. 309 (3): 657-670; 2001); Chothia (Al-Lazikani et al., 1997 Journal of Molecular Biology 273:927-948); and Contact (Maccallum et al., 1996 Journal of Molecular Biology 262:732-745).

An “antibody” and “antibodies” and related terms used herein refers to an intact immunoglobulin or to an antigen binding portion thereof (or an antigen binding fragment thereof) that binds specifically to an antigen. Antigen binding portions (or the antigen binding fragment) may be produced by recombinant DNA techniques or by enzymatic or chemical cleavage of intact antibodies. Antigen binding portions (or antigen binding fragments) include, inter alia, Fab, Fab′, F(ab′)₂, Fv, domain antibodies (dAbs), and complementarity determining region (CDR) fragments, single-chain antibodies (scFv), chimeric antibodies, diabodies, triabodies, tetrabodies, and polypeptides that contain at least a portion of an immunoglobulin that is sufficient to confer specific antigen binding to the polypeptide.

Antibodies include recombinantly produced antibodies and antigen binding portions. Antibodies include non-human, chimeric, humanized and fully human antibodies. Antibodies include monospecific, multispecific (e.g., bispecific, trispecific and higher order specificities). Antibodies include tetrameric antibodies, light chain monomers, heavy chain monomers, light chain dimers, heavy chain dimers. Antibodies include F(ab′)₂ fragments, Fab′ fragments and Fab fragments. Antibodies include single domain antibodies, monovalent antibodies, single chain antibodies, single chain variable fragment (scFv), camelized antibodies, affibodies, disulfide-linked Fvs (sdFv), anti-idiotypic antibodies (anti-Id), minibodies. Antibodies include monoclonal and polyclonal populations. Anti-ROR1 antibodies are described herein.

An “antigen binding domain,” “antigen binding region,” or “antigen binding site” and other related terms used herein refer to a portion of an antigen binding protein that contains amino acid residues (or other moieties) that interact with an antigen and contribute to the antigen binding protein's specificity and affinity for the antigen. For an antibody that specifically binds to its antigen, this will include at least part of at least one of its CDR domains. Antigen binding domains from anti-ROR1 antibodies are described herein.

The terms “specific binding”, “specifically binds” or “specifically binding” and other related terms, as used herein in the context of an antibody or antigen binding protein or antibody fragment, refer to non-covalent or covalent preferential binding to an antigen relative to other molecules or moieties (e.g., an antibody specifically binds to a particular antigen relative to other available antigens). In embodiments, an antibody specifically binds to a target antigen if it binds to the antigen with a dissociation constant Kp of 10⁻⁵ M or less, or 10⁻⁶ M or less, or 10⁻⁷ M or less, or 10⁻⁸ M or less, or 10⁻⁹ M or less, or 10⁻¹⁰ M or less. Anti-ROR1 antibodies that specifically bind ROR1 are described herein.

In embodiments, a dissociation constant (K_{D}) can be measured using a BIACORE surface plasmon resonance (SPR) assay. Surface plasmon resonance refers to an optical phenomenon that allows for the analysis of real-time interactions by detection of alterations in protein concentrations within a biosensor matrix, for example using the BIACORE system (Biacore Life Sciences division of GE Healthcare, Piscataway, NJ).

An “epitope” and related terms as used herein refers to a portion of an antigen that is bound by an antigen binding protein (e.g., by an antibody or an antigen binding portion thereof). An epitope can comprise portions of two or more antigens that are bound by an antigen binding protein. An epitope can comprise non-contiguous portions of an antigen or of two or more antigens (e.g., amino acid residues that are not contiguous in an antigen's primary sequence but that, in the context of the antigen's tertiary and quaternary structure, are near enough to each other to be bound by an antigen binding protein). Generally, the variable regions, particularly the CDRs, of an antibody interact with the epitope. Anti-ROR1 antibodies, and antigen binding proteins thereof, that bind an epitope of a ROR1 polypeptide are described herein.

With respect to antibodies, the term “antagonist” and “antagonistic” refers to a blocking antibody that binds its cognate target antigen and inhibits or reduces the biological activity of the bound antigen. The term “agonist” or “agonistic” refers to an antibody that binds its cognate target antigen in a manner that mimics the binding of the physiological ligand which causes antibody-mediated downstream signaling.

An “antibody fragment”, “antibody portion”, “antigen-binding fragment of an antibody”, or “antigen-binding portion of an antibody” and other related terms used herein refer to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; Fd; and Fv fragments, as well as dAb; diabodies; linear antibodies; single-chain antibody molecules (e.g. scFv); polypeptides that contain at least a portion of an antibody that is sufficient to confer specific antigen binding to the polypeptide. Antigen binding portions of an antibody may be produced by recombinant DNA techniques or by enzymatic or chemical cleavage of intact antibodies. Antigen binding portions include, inter alia, Fab, Fab′, F(ab′)₂, Fv, domain antibodies (dAbs), and complementarity determining region (CDR) fragments, chimeric antibodies, diabodies, triabodies, tetrabodies, and polypeptides that contain at least a portion of an immunoglobulin that is sufficient to confer antigen binding properties to the antibody fragment. Antigen-binding fragments of anti-ROR1 antibodies are described herein.

The terms “Fab”, “Fab fragment” and other related terms refers to a monovalent fragment comprising a variable light chain region (V_{L}), constant light chain region (C_{L}), variable heavy chain region (V_{H}), and first constant region (C_{H1}). A Fab is capable of binding an antigen. An F(ab′)₂ fragment is a bivalent fragment comprising two Fab fragments linked by a disulfide bridge at the hinge region. A F(Ab′)₂ has antigen binding capability. An Fd fragment comprises V_{H} and C_{H1} regions. An Fv fragment comprises V_{L} and V_{H} regions. An Fv can bind an antigen. A dAb fragment has a V_{H} domain, a V_{L} domain, or an antigen-binding fragment of a V_{H} or V_{L} domain (U.S. Pat. Nos. 6,846,634 and 6,696,245; U.S. published Application Nos. 2002/02512, 2004/0202995, 2004/0038291, 2004/0009507, 2003/0039958; and Ward et al., Nature 341:544-546, 1989). Fab fragments comprising antigen binding portions from anti-ROR1 antibodies are described herein.

A single-chain antibody (scFv) is an antibody in which a V_{L} and a V_{H} region are joined via a linker (e.g., a synthetic sequence of amino acid residues) to form a continuous protein chain. Preferably the linker is long enough to allow the protein chain to fold back on itself and form a monovalent antigen binding site (see, e.g., Bird et al., 1988, Science 242:423-26 and Huston et al., 1988, Proc. Natl. Acad. Sci. USA 85:5879-83). Single chain antibodies comprising antigen binding portions from anti-ROR1 antibodies are described herein.

Diabodies are bivalent antibodies comprising two polypeptide chains, wherein each polypeptide chain comprises V_{H} and V_{L} domains joined by a linker that is too short to allow for pairing between two domains on the same chain, thus allowing each domain to pair with a complementary domain on another polypeptide chain (see, e.g., Holliger et al., 1993, Proc. Natl. Acad. Sci. USA 90:6444-48, and Poljak et al., 1994, Structure 2:1121-23). If the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different. Diabody, tribody and tetrabody constructs can be prepared using antigen binding portions from any of the anti-ROR1 antibodies described herein.

The term “human antibody” refers to antibodies that have one or more variable and constant regions derived from human immunoglobulin sequences. In embodiments, all of the variable and constant domains are derived from human immunoglobulin sequences (e.g., a fully human antibody). These antibodies may be prepared in a variety of ways, examples of which are described below, including through recombinant methodologies or through immunization with an antigen of interest of a mouse that is genetically modified to express antibodies derived from human heavy and/or light chain-encoding genes. Fully human anti-ROR1 antibodies and antigen binding proteins thereof are described herein.

A “humanized” antibody refers to an antibody having a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In embodiments, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293.

The term “chimeric antibody” and related terms used herein refers to an antibody that contains one or more regions from a first antibody and one or more regions from one or more other antibodies. In embodiments, one or more of the CDRs are derived from a human antibody. In another embodiment, all of the CDRs are derived from a human antibody. In another embodiment, the CDRs from more than one human antibody are mixed and matched in a chimeric antibody. For instance, a chimeric antibody may comprise a CDR1 from the light chain of a first human antibody, a CDR2 and a CDR3 from the light chain of a second human antibody, and the CDRs from the heavy chain from a third antibody. In another example, the CDRs originate from different species such as human and mouse, or human and rabbit, or human and goat. One skilled in the art will appreciate that other combinations are possible.

Further, the framework regions may be derived from one of the same antibodies, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody(-ies) from another species or belonging to another antibody class or subclass. Also included are fragments of such antibodies that exhibit the desired biological activity (i.e., the ability to specifically bind a target antigen). Chimeric antibodies can be prepared from portions of any of the anti-ROR1 antibodies described herein.

As used herein, the term “variant” polypeptides and “variants” of polypeptides refers to a polypeptide comprising an amino acid sequence with one or more amino acid residues inserted into, deleted from and/or substituted into the amino acid sequence relative to a reference polypeptide sequence. Polypeptide variants include fusion proteins. In the same manner, a variant polynucleotide comprises a nucleotide sequence with one or more nucleotides inserted into, deleted from and/or substituted into the nucleotide sequence relative to another polynucleotide sequence. Polynucleotide variants include fusion polynucleotides.

As used herein, the term “derivative” of a polypeptide is a polypeptide (e.g., an antibody) that has been chemically modified, e.g., via conjugation to another chemical moiety such as, for example, polyethylene glycol, albumin (e.g., human serum albumin), phosphorylation, and glycosylation. Unless otherwise indicated, the term “antibody” includes, in addition to antibodies comprising two full-length heavy chains and two full-length light chains, derivatives, variants, fragments, and muteins thereof, examples of which are described below.

The term “hinge” refers to an amino acid segment that is generally found between two domains of a protein and may allow for flexibility of the overall construct and movement of one or both of the domains relative to one another. Structurally, a hinge region comprises from about 10 to about 100 amino acids, e.g., from about 15 to about 75 amino acids, from about 20 to about 50 amino acids, or from about 30 to about 60 amino acids. In embodiments, the hinge region is 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 amino acids in length. The hinge region can be derived from a hinge region of a naturally-occurring protein, such as a CD8 hinge region or a fragment thereof, a CD8a hinge region, or a fragment thereof, a hinge region of an antibody (e.g., IgG, IgA, IgM, IgE, or IgD antibodies), or a hinge region that joins the constant domains CH1 and CH2 of an antibody. The hinge region can be derived from an antibody and may or may not comprise one or more constant regions of the antibody, or the hinge region comprises the hinge region of an antibody and the CH3 constant region of the antibody, or the hinge region comprises the hinge region of an antibody and the CH2 and CH3 constant regions of the antibody, or the hinge region is a non-naturally occurring peptide, or the hinge region is disposed between the C-terminus of the scFv and the N-terminus of the transmembrane domain. In embodiments, the hinge region comprises any one or any combination of two or more regions comprising an upper, core or lower hinge sequences from an IgG1, IgG2, IgG3 or IgG4 immunoglobulin molecule. In embodiments, the hinge region comprises an IgG1 upper hinge sequence EPKSCDKTHT. In embodiments, the hinge region comprises an IgG1 core hinge sequence CPXCP, wherein X is P, R or S. In embodiments, the hinge region comprises a lower hinge/CH2 sequence APELLGGP. In embodiments, the hinge is joined to an Fc region (CH2) having the amino acid sequence SVFLFPPKPKDT. In embodiments, the hinge region includes the amino acid sequence of an upper, core and lower hinge and comprises EPKSCDKTHTCPPCPAPELLGGP. In embodiments, the hinge region comprises one, two, three or more cysteines that can form at least one, two, three or more interchain disulfide bonds.

The term “Fc” or “Fc region” as used herein refers to the portion of an antibody heavy chain constant region beginning in or after the hinge region and ending at the C-terminus of the heavy chain. The Fc region comprises at least a portion of the CH2 and CH3 regions and may, or may not, include a portion of the hinge region. Two polypeptide chains each carrying a half Fc region can dimerize to form a full Fc domain. An Fc domain can bind Fc cell surface receptors and some proteins of the immune complement system. An Fc region can bind a complement component C1q. An Fc domain exhibits effector function, including any one or any combination of two or more activities including complement-dependent cytotoxicity (CDC), antibody-dependent cell-mediated cytotoxicity (ADCC), antibody-dependent phagocytosis (ADP), opsonization and/or cell binding. An Fc domain can bind an Fc receptor, including FcγRI (e.g., CD64), FcγRII (e.g, CD32) and/or FcγRIII (e.g., CD16a). In embodiments, the Fc region can include a mutation that increases or decreases any one or any combination of these functions.

The term “labeled antibody” or related terms as used herein refers to antibodies and their antigen binding portions thereof that are unlabeled or joined to a detectable label or moiety for detection, wherein the detectable label or moiety is radioactive, colorimetric, antigenic, enzymatic, a detectable bead (such as a magnetic or electrodense (e.g., gold) bead), biotin, streptavidin or protein A. A variety of labels can be employed, including, but not limited to, radionuclides, fluorescers, enzymes, enzyme substrates, enzyme cofactors, enzyme inhibitors and ligands (e.g., biotin, haptens). Any of the anti-ROR1 antibodies described herein can be unlabeled or can be joined to a detectable label or moiety.

The “percent identity” or “percent homology” and related terms used herein refers to a quantitative measurement of the similarity between two polypeptide or between two polynucleotide sequences. The percent identity between two polypeptide sequences is a function of the number of identical amino acids at aligned positions that are shared between the two polypeptide sequences, taking into account the number of gaps, and the length of each gap, which may need to be introduced to optimize alignment of the two polypeptide sequences. In a similar manner, the percent identity between two polynucleotide sequences is a function of the number of identical nucleotides at aligned positions that are shared between the two polynucleotide sequences, taking into account the number of gaps, and the length of each gap, which may need to be introduced to optimize alignment of the two polynucleotide sequences. A comparison of the sequences and determination of the percent identity between two polypeptide sequences, or between two polynucleotide sequences, may be accomplished using a mathematical algorithm. For example, the “percent identity” or “percent homology” of two polypeptide or two polynucleotide sequences may be determined by comparing the sequences using the GAP computer program (a part of the GCG Wisconsin Package, version 10.3 (Accelrys, San Diego, Calif.)) using its default parameters. Expressions such as “comprises a sequence with at least X % identity to Y” with respect to a test sequence mean that, when aligned to sequence Y as described above, the test sequence comprises residues identical to at least X % of the residues of Y.

In embodiments, the amino acid sequence of a test antibody may be similar but not necessarily identical to any of the amino acid sequences of the polypeptides that make up any of the anti-ROR1 antibodies, or antigen binding protein thereof, described herein. The similarities between the test antibody and the polypeptides can be at least 95%, or at or at least 96% identical, or at least 97% identical, or at least 98% identical, or at least 99% identical, to any of the polypeptides that make up any of the anti-ROR1 antibodies, or antigen binding protein thereof, described herein. In embodiments, similar polypeptides can contain amino acid substitutions within a heavy and/or light chain. In embodiments, the amino acid substitutions comprise one or more conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24:307-331, herein incorporated by reference in its entirety. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine.

Antibodies can be obtained from sources such as serum or plasma that contain immunoglobulins having varied antigenic specificity. If such antibodies are subjected to affinity purification, they can be enriched for a particular antigenic specificity. Such enriched preparations of antibodies usually are made of less than about 10% antibody having specific binding activity for the particular antigen. Subjecting these preparations to several rounds of affinity purification can increase the proportion of antibody having specific binding activity for the antigen. Antibodies prepared in this manner are often referred to as “monospecific.” Monospecfic antibody preparations can be made up of about 10%, 20%, 30%, 40%, 50%, 60%, 70%, 75%, 80%, 85%, 90%, 95%, 97%, 99%, or 99.9% antibody having specific binding activity for the particular antigen. Antibodies can be produced using recombinant nucleic acid technology as described below.

A “vector” and related terms used herein refers to a nucleic acid molecule (e.g., DNA or RNA) which can be operably linked to foreign genetic material (e.g., nucleic acid transgene). Vectors can be used as a vehicle to introduce foreign genetic material into a cell (e.g., host cell). Vectors can include at least one restriction endonuclease recognition sequence for insertion of the transgene into the vector. Vectors can include at least one gene sequence that confers antibiotic resistance or a selectable characteristic to aid in selection of host cells that harbor a vector-transgene construct. Vectors can be single-stranded or double-stranded nucleic acid molecules. Vectors can be linear or circular nucleic acid molecules. A donor nucleic acid used for gene editing methods employing zinc finger nuclease, TALEN or CRISPR/Cas can be a type of a vector. One type of vector is a “plasmid,” which refers to a linear or circular double stranded extrachromosomal DNA molecule which can be linked to a transgene, and is capable of replicating in a host cell, and transcribing and/or translating the transgene. A viral vector typically contains viral RNA or DNA backbone sequences which can be linked to the transgene. The viral backbone sequences can be modified to disable infection but retain insertion of the viral backbone and the co-linked transgene into a host cell genome. Examples of viral vectors include retroviral, lentiviral, adenoviral, adeno-associated, baculoviral, papovaviral, vaccinia viral, herpes simplex viral and Epstein Barr viral vectors. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome.

An “expression vector” is a type of vector that can contain one or more regulatory sequences, such as inducible and/or constitutive promoters and enhancers. Expression vectors can include ribosomal binding sites and/or polyadenylation sites. Regulatory sequences direct transcription, or transcription and translation, of a transgene linked to the expression vector which is transduced into a host cell. The regulatory sequence(s) can control the level, timing and/or location of expression of the transgene. The regulatory sequence can, for example, exert its effects directly on the transgene, or through the action of one or more other molecules (e.g., polypeptides that bind to the regulatory sequence and/or the nucleic acid). Regulatory sequences can be part of a vector. Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-3606. An expression vector can comprise nucleic acids that encode at least a portion of any of the anti-ROR1 antibodies described herein.

A transgene is “operably linked” to a vector when there is linkage between the transgene and the vector to permit functioning or expression of the transgene sequences contained in the vector. In embodiments, a transgene is “operably linked” to a regulatory sequence when the regulatory sequence affects the expression (e.g., the level, timing, or location of expression) of the transgene.

The terms “transfected” or “transformed” or “transduced” or other related terms used herein refer to a process by which exogenous nucleic acid (e.g., transgene) is transferred or introduced into a host cell. A “transfected” or “transformed” or “transduced” host cell is one which has been transfected, transformed or transduced with exogenous nucleic acid (transgene). The host cell includes the primary subject cell and its progeny. Exogenous nucleic acids encoding at least a portion of any of the anti-ROR1 antibodies described herein can be introduced into a host cell. Expression vectors comprising at least a portion of any of the anti-ROR1 antibodies described herein can be introduced into a host cell, and the host cell can express polypeptides comprising at least a portion of the anti-ROR1 antibody.

The terms “host cell” or “or a population of host cells” or related terms as used herein refer to a cell (or a population thereof) into which foreign (exogenous or transgene) nucleic acids have been introduced. The foreign nucleic acids can include an expression vector operably linked to a transgene, and the host cell can be used to express the nucleic acid and/or polypeptide encoded by the foreign nucleic acid (transgene). A host cell (or a population thereof) can be a cultured cell or can be extracted from a subject. The host cell (or a population thereof) includes the primary subject cell and its progeny without any regard for the number of passages. Progeny cells may or may not harbor identical genetic material compared to the parent cell. Host cells encompass progeny cells. In embodiments, a host cell describes any cell (including its progeny) that has been modified, transfected, transduced, transformed, and/or manipulated in any way to express an antibody, as disclosed herein. In one example, the host cell (or population thereof) can be introduced with an expression vector operably linked to a nucleic acid encoding the desired antibody, or an antigen binding portion thereof, described herein. Host cells and populations thereof can harbor an expression vector that is stably integrated into the host's genome or can harbor an extrachromosomal expression vector. In embodiments, host cells and populations thereof can harbor an extrachromosomal vector that is present after several cell divisions or is present transiently and is lost after several cell divisions.

A host cell can be a prokaryote, for example, E. coli, or it can be a eukaryote, for example, a single-celled eukaryote (e.g., a yeast or other fungus), a plant cell (e.g., a tobacco or tomato plant cell), an mammalian cell (e.g., a human cell, a monkey cell, a hamster cell, a rat cell, a mouse cell, or an insect cell) or a hybridoma. In embodiments, a host cell can be introduced with an expression vector operably linked to a nucleic acid encoding a desired antibody thereby generating a transfected/transformed host cell which is cultured under conditions suitable for expression of the antibody by the transfected/transformed host cell, and optionally recovering the antibody from the transfected/transformed host cells (e.g., recovery from host cell lysate) or recovery from the culture medium. In embodiments, host cells comprise non-human cells including CHO, BHK, NS0, SP2/0, and YB2/0. In embodiments, host cells comprise human cells including HEK293, HT-1080, Huh-7 and PER.C6. Examples of host cells include the COS-7 line of monkey kidney cells (ATCC CRL 1651) (see Gluzman et al., 1981, Cell 23:175), L cells, C127 cells, 3T3 cells (ATCC CCL 163), Chinese hamster ovary (CHO) cells or their derivatives such as Veggie CHO and related cell lines which grow in serum-free media (see Rasmussen et al., 1998, Cytotechnology 28:31) or CHO strain DX-B 11, which is deficient in DHFR (see Urlaub et al., 1980, Proc. Natl. Acad. Sci. USA 77:4216-20), HeLa cells, BHK (ATCC CRL 10) cell lines, the CV1/EBNA cell line derived from the African green monkey kidney cell line CV1 (ATCC CCL 70) (see McMahan et al., 1991, EMBO J. 10:2821), human embryonic kidney cells such as 293, 293 EBNA or MSR 293, human epidermal A431 cells, human Colo 205 cells, other transformed primate cell lines, normal diploid cells, cell strains derived from in vitro culture of primary tissue, primary explants, HL-60, U937, HaK or Jurkat cells. In embodiments, host cells include lymphoid cells such as Y0, NS0 or Sp20. In embodiments, a host cell is a mammalian host cell, but is not a human host cell. Typically, a host cell is a cultured cell that can be transformed or transfected with a polypeptide-encoding nucleic acid, which can then be expressed in the host cell. The phrase “transgenic host cell” or “recombinant host cell” can be used to denote a host cell that has been transformed or transfected with a nucleic acid to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but also to the progeny or potential progeny of such a cell. Because certain modifications may occur in succeeding generations due to, e.g., mutation or environmental influence, such progeny may not, in fact, be identical to the parent cell, but are still included within the scope of the term as used herein.

Polypeptides of the present disclosure (e.g., antibodies and antigen binding proteins) can be produced using any methods known in the art. In one example, the polypeptides are produced by recombinant nucleic acid methods by inserting a nucleic acid sequence (e.g., DNA) encoding the polypeptide into a recombinant expression vector which is introduced into a host cell and expressed by the host cell under conditions promoting expression.

General techniques for recombinant nucleic acid manipulations are described for example in Sambrook et al., in Molecular Cloning: A Laboratory Manual, Vols. 1-3, Cold Spring Harbor Laboratory Press, 2 ed., 1989, or F. Ausubel et al., in Current Protocols in Molecular Biology (Green Publishing and Wiley-Interscience: New York, 1987) and periodic updates, herein incorporated by reference in their entireties. The nucleic acid (e.g., DNA) encoding the polypeptide is operably linked to an expression vector carrying one or more suitable transcriptional or translational regulatory elements derived from mammalian, viral, or insect genes. Such regulatory elements include a transcriptional promoter, an optional operator sequence to control transcription, a sequence encoding suitable mRNA ribosomal binding sites, and sequences that control the termination of transcription and translation. The expression vector can include an origin or replication that confers replication capabilities in the host cell. The expression vector can include a gene that confers selection to facilitate recognition of transgenic host cells (e.g., transformants).

The recombinant DNA can also encode any type of protein tag sequence that may be useful for purifying the protein. Examples of protein tags include but are not limited to a histidine tag, a FLAG tag, a myc tag, an HA tag, or a GST tag. Appropriate cloning and expression vectors for use with bacterial, fungal, yeast, and mammalian cellular hosts can be found in Cloning Vectors: A Laboratory Manual, (Elsevier, N.Y., 1985).

The expression vector construct can be introduced into the host cell using a method appropriate for the host cell. A variety of methods for introducing nucleic acids into host cells are known in the art, including, but not limited to, electroporation; transfection employing calcium chloride, rubidium chloride, calcium phosphate, DEAE-dextran, or other substances; viral transfection; non-viral transfection; microprojectile bombardment; lipofection; and infection (e.g., where the vector is an infectious agent). Suitable host cells include prokaryotes, yeast, mammalian cells, or bacterial cells.

Suitable bacteria include gram negative or gram positive organisms, for example, F. coli or Bacillus spp. Yeast, preferably from the Saccharomyces species, such as S. cerevisiae, may also be used for production of polypeptides. Various mammalian or insect cell culture systems can also be employed to express recombinant proteins. Baculovirus systems for production of heterologous proteins in insect cells are reviewed by Luckow and Summers, (Bio/Technology, 6:47, 1988). Examples of suitable mammalian host cell lines include endothelial cells, COS-7 monkey kidney cells, CV-1, L cells, C127, 3T3, Chinese hamster ovary (CHO), human embryonic kidney cells, HeLa, 293, 293T, and BHK cell lines. Purified polypeptides are prepared by culturing suitable host/vector systems to express the recombinant proteins. For many applications, the small size of many of the polypeptides disclosed herein would make expression in E. coli as the preferred method for expression. The protein is then purified from culture media or cell extracts. Any of the anti-ROR1 antibodies, or antigen binding protein thereof, can be expressed by transgenic host cells.

Antibodies and antigen binding proteins disclosed herein can also be produced using cell-translation systems. For such purposes the nucleic acids encoding the polypeptide must be modified to allow in vitro transcription to produce mRNA and to allow cell-free translation of the mRNA in the particular cell-free system being utilized (eukaryotic such as a mammalian or yeast cell-free translation system or prokaryotic such as a bacterial cell-free translation system.

Nucleic acids encoding any of the various polypeptides disclosed herein may be synthesized chemically. Codon usage may be selected so as to improve expression in a cell. Such codon usage will depend on the cell type selected. Specialized codon usage patterns have been developed for E. coli and other bacteria, as well as mammalian cells, plant cells, yeast cells and insect cells. See for example: Mayfield et al., Proc. Natl. Acad. Sci. USA. 2003 100 (2): 438-42; Sinclair et al. Protein Expr. Purif. 2002 (1): 96-105; Connell N D. Curr. Opin. Biotechnol. 2001 12 (5): 446-9; Makrides et al. Microbiol. Rev. 1996 60 (3): 512-38; and Sharp et al. Yeast. 1991 7 (7): 657-78.

Antibodies and antigen binding proteins described herein can also be produced by chemical synthesis (e.g., by the methods described in Solid Phase Peptide Synthesis, 2nd ed., 1984, The Pierce Chemical Co., Rockford, Ill.). Modifications to the protein can also be produced by chemical synthesis.

Antibodies and antigen binding proteins described herein can be purified by isolation/purification methods for proteins generally known in the field of protein chemistry. Non-limiting examples include extraction, recrystallization, salting out (e.g., with ammonium sulfate or sodium sulfate), centrifugation, dialysis, ultrafiltration, adsorption chromatography, ion exchange chromatography, hydrophobic chromatography, normal phase chromatography, reversed-phase chromatography, gel filtration, gel permeation chromatography, affinity chromatography, electrophoresis, countercurrent distribution or any combinations of these. After purification, polypeptides may be exchanged into different buffers and/or concentrated by any of a variety of methods known to the art, including, but not limited to, filtration and dialysis.

The purified antibodies and antigen binding proteins described herein are preferably at least 65% pure, at least 75% pure, at least 85% pure, more preferably at least 95% pure, and most preferably at least 98% pure. Regardless of the exact numerical value of the purity, the polypeptide is sufficiently pure for use as a pharmaceutical product. Any of the anti-ROR1 antibodies, or antigen binding protein thereof, described herein can be expressed by transgenic host cells and then purified to about 65-98% purity or high level of purity using any art-known method.

In certain embodiments, the antibodies and antigen binding proteins herein can further comprise post-translational modifications. Exemplary post-translational protein modifications include phosphorylation, acetylation, methylation, ADP-ribosylation, ubiquitination, glycosylation, carbonylation, sumoylation, biotinylation or addition of a polypeptide side chain or of a hydrophobic group. As a result, the modified polypeptides may contain non-amino acid elements, such as lipids, poly- or mono-saccharide, and phosphates. A preferred form of glycosylation is sialylation, which conjugates one or more sialic acid moieties to the polypeptide. Sialic acid moieties improve solubility and serum half-life while also reducing the possible immunogenicity of the protein. See Raju et al. Biochemistry. 2001 31; 40 (30): 8868-76.

In embodiments, the antibodies and antigen binding proteins described herein can be modified to become soluble polypeptides which comprises linking the Antibodies and antigen binding proteins to non-proteinaceous polymers. In embodiments, the non-proteinaceous polymer comprises polyethylene glycol (“PEG”), polypropylene glycol, or polyoxyalkylenes, in the manner as set forth in U.S. Pat. Nos. 4,640,835; 4,496,689; 4,301, 144; 4,670,417; 4,791,192 or 4,179,337.

PEG is a water soluble polymer that is commercially available or can be prepared by ring-opening polymerization of ethylene glycol according to methods well known in the art (Sandler and Karo, Polymer Synthesis, Academic Press, New York, Vol. 3, pages 138-161). The term “PEG” is used broadly to encompass any polyethylene glycol molecule, without regard to size or to modification at an end of the PEG, and can be represented by the formula: X—O(CH₂CH₂O)ₙ—CH₂CH₂OH (1), where n is 20 to 2300 and X is H or a terminal modification, e.g., a C₁₋₄ alkyl. In embodiments, the PEG terminates on one end with hydroxy or methoxy, i.e., X is H or CH₃ (“methoxy PEG”). A PEG can contain further chemical groups which are necessary for binding reactions; which results from the chemical synthesis of the molecule; or which is a spacer for optimal distance of parts of the molecule. In addition, such a PEG can consist of one or more PEG side-chains which are linked together. PEGs with more than one PEG chain are called multiarmed or branched PEGs. Branched PEGs can be prepared, for example, by the addition of polyethylene oxide to various polyols, including glycerol, pentaerythriol, and sorbitol. For example, a four-armed branched PEG can be prepared from pentaerythriol and ethylene oxide. Branched PEG are described in, for example, EP-A 0 473 084 and U.S. Pat. No. 5,932,462. One form of PEGs includes two PEG side-chains (PEG2) linked via the primary amino groups of a lysine (Monfardini et al., Bioconjugate Chem. 6 (1995) 62-69).

The serum clearance rate of PEG-modified polypeptide may be modulated (e.g., increased or decreased) by about 10%, 20%, 30%, 40%, 50%, 60%, 70%, 80%, or even 90%, relative to the clearance rate of the unmodified antibodies and antigen binding proteins binding polypeptides. The PEG-modified antibodies and antigen binding proteins may have a half-life (t_{1/2}) which is enhanced relative to the half-life of the unmodified polypeptide. The half-life of PEG-modified polypeptide may be enhanced by at least 10%, 20%, 30%, 40%, 50%, 60%, 70%, 80%, 90%, 100%, 125%, 150%, 175%, 200%, 250%, 300%, 400% or 500%, or even by 1000% relative to the half-life of the unmodified antibodies and antigen binding proteins. In some embodiments, the protein half-life is determined in vitro, such as in a buffered saline solution or in serum. In other embodiments, the protein half-life is an in vivo half-life, such as the half-life of the protein in the serum or other bodily fluid of an animal.

The present disclosure provides therapeutic compositions comprising any of the anti-ROR1 antibodies, or antigen binding protein thereof, described herein in an admixture with a pharmaceutically-acceptable excipient. An excipient encompasses carriers, stabilizers and excipients. Excipients of pharmaceutically acceptable excipients includes for example inert diluents or fillers (e.g., sucrose and sorbitol), lubricating agents, glidants, and anti-adhesives (e.g., magnesium stearate, zinc stearate, stearic acid, silicas, hydrogenated vegetable oils, or talc). Additional examples include buffering agents, stabilizing agents, preservatives, non-ionic detergents, anti-oxidants and isotonifiers.

Therapeutic compositions and methods for preparing them are well known in the art and are found, for example, in “Remington: The Science and Practice of Pharmacy” (20th ed., ed. A. R. Gennaro A R., 2000, Lippincott Williams & Wilkins, Philadelphia, Pa.). Therapeutic compositions can be formulated for parenteral administration may, and can for example, contain excipients, sterile water, saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the antibody (or antigen binding protein thereof) described herein. Nanoparticulate formulations (e.g., biodegradable nanoparticles, solid lipid nanoparticles, liposomes) may be used to control the biodistribution of the antibody (or antigen binding protein thereof). Other potentially useful parenteral delivery systems include ethylene-vinyl acetate copolymer particles, osmotic pumps, implantable infusion systems, and liposomes. The concentration of the antibody (or antigen binding protein thereof) in the formulation varies depending upon a number of factors, including the dosage of the drug to be administered, and the route of administration.

Any of the anti-ROR1 antibodies (or antigen binding portions thereof) may be administered as a pharmaceutically acceptable salt, such as non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, and the like. In one example, the antibody (or antigen binding portions thereof) is formulated in the presence of sodium acetate to increase thermal stability.

Any of the anti-ROR1 antibodies (or antigen binding portions thereof) may be formulated for oral use include tablets containing the active ingredient(s) in a mixture with non-toxic pharmaceutically acceptable excipients. Formulations for oral use may also be provided as chewable tablets, or as hard gelatin capsules wherein the active ingredient is mixed with an inert solid diluent, or as soft gelatin capsules wherein the active ingredient is mixed with water or an oil medium.

The term “subject” as used herein refers to human and non-human animals, including vertebrates, mammals and non-mammals. In embodiments, the subject can be human, non-human primates, simian, ape, murine (e.g., mice and rats), bovine, porcine, equine, canine, feline, caprine, lupine, ranine or piscine.

The term “administering”, “administered” and grammatical variants refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods. Any of the anti-ROR1 antibodies described herein (or antigen binding protein thereof) can be administered to a subject using art-known methods and delivery routes.

The terms “effective amount”, “therapeutically effective amount” or “effective dose” or related terms may be used interchangeably and refer to an amount of antibody or an antigen binding protein (e.g., any of the anti-ROR1 antibodies described herein or antigen binding protein thereof) that when administered to a subject, is sufficient to effect a measurable improvement or prevention of a disease or disorder associated with tumor or cancer antigen expression. Therapeutically effective amounts of antibodies provided herein, when used alone or in combination, will vary depending upon the relative activity of the antibodies and combinations (e.g., in inhibiting cell growth) and depending upon the subject and disease condition being treated, the weight and age and sex of the subject, the severity of the disease condition in the subject, the manner of administration and the like, which can readily be determined by one of ordinary skill in the art.

In embodiments, a therapeutically effective amount will depend on certain aspects of the subject to be treated and the disorder to be treated and may be ascertained by one skilled in the art using known techniques. In general, the polypeptide is administered at about 0.01 g/kg to about 50 mg/kg per day, preferably 0.01 mg/kg to about 30 mg/kg per day, most preferably 0.1 mg/kg to about 20 mg/kg per day. The polypeptide may be administered daily (e.g., once, twice, three times, or four times daily) or preferably less frequently (e.g., weekly, every two weeks, every three weeks, monthly, or quarterly). In addition, as is known in the art, adjustments for age as well as the body weight, general health, sex, diet, time of administration, drug interaction, and the severity of the disease may be necessary.

The present disclosure provides methods for treating a subject having a disease associated with expression or over-expression of ROR1. The disease comprises cancer or tumor cells expressing the tumor-associated antigens. In embodiments, the cancer or tumor includes the chronic lymphocytic leukemia (CLL), breast cancer, lung cancer, gastric cancer, melanoma, colon cancer, renal cell carcinoma, and lymphomas.

High proportions of human cancers express ROR1. For example, Zhang et al. showed that 54% ovarian cancers, 57% colon cancers, 77% lung cancers, 90% lymphomas, 89% skin cancers, 83% pancreatic cancers, 73% testicular cancers, 43% bladder cancers, 96% uterus cancers, 90% prostate cancers, and 83% adrenal cancers that they examined had moderate-to-strong staining with the anti-ROR1 antibody 4A5 (Zhang et al., 2012, Am. J. Pathol., 181 (6), 1903-1910). Daneshmanesh et al. similarly found near universal expression of ROR1 in CLL and hairy cell leukemia (HCL) and varying degrees of expression in other lymphoid cancers such as mantle cell lymphoma (MCL), diffuse large B-cell lymphoma (DLBCL)/marginal zone lymphoma (MZL), follicular lymphoma (FL), chronic myeloid leukemia (CML), acute myeloid lymphoma (AML), and myeloma (Daneshmanesh et al., 2013, Leuk. Lymphoma 54 (4), 843-850). Additionally, substantial proportions of patients with hepatocellular cancers (HCC) or non-small-cell lung cancer (NSCLC) are ROR1-positive. Further, it has been shown that ROR1 expression increases in aggressive cancers and correlates with poor prognosis.

In embodiments, the cancer is chronic lymphocytic leukemia (CLL), T-cell leukemia (TCL), mantle cell lymphoma (MCL), diffuse large B-cell lymphoma (DLBCL), Burkitt's lymphoma, multiple myeloma (MM), marginal zone lymphoma (MZL), small lymphocytic lymphoma (SLL), or a non-Hodgkin lymphoma (NHL) that has undergone Richter's transformation. In embodiments, the cancer is non-small cell lung cancer (NSCLC), hepatocellular carcinoma, pancreatic cancer, osteosarcoma, head and neck cancer, ovarian cancer, breast cancer, or triple negative breast cancer (TNBC). In embodiments, the antibodies are for use in treating hematological malignancies. In embodiments, the antibodies are for use in treating solid tumors. The cancer to be treated may be selected from, e.g., lymphoma, small lymphocytic lymphoma, marginal zone lymphoma, marginal cell B-cell lymphoma, Burkitt's lymphoma, mantle cell lymphoma, diffuse large B-cell lymphoma, a non-Hodgkin lymphoma that has undergone Richter's transformation, chronic lymphocytic leukemia, T cell leukemia, osteosarcoma, renal cell carcinoma, hepatocellular carcinoma, colon cancer, colorectal cancer, breast cancer, epithelial squamous cell cancer, melanoma, myeloma, multiple myeloma, stomach cancer, brain cancer, lung cancer, non-small cell lung cancer, pancreatic cancer, cervical cancer, ovarian cancer, liver cancer, bladder cancer, prostate cancer, testicular cancer, thyroid cancer, and head and neck cancer. In embodiments, the cancer to be treated can be a cancer that is refractory to other therapeutics (for example, triple negative breast cancer). In embodiments, the cancer can be a metastatic cancer, a refractory cancer or a recurrent cancer.

The present disclosure provides ROR1 binding proteins, particularly anti-ROR1 antibodies, or antigen binding portions thereof, that specifically bind ROR1 and uses thereof. In embodiments, the ROR1 binding proteins bind an epitope of ROR1. Tyrosine-protein kinase transmembrane receptor ROR1 also known as neurotrophic tyrosine kinase, receptor-related 1 (NTRKR1) (e.g., UniProt Q01973-1).

Various aspects of the anti-ROR1 antibodies relate to antibody fragments, single-chain antibodies, pharmaceutical compositions, nucleic acids, recombinant expression vectors, host cells, and methods for preparing and using such anti-ROR1 antibodies. Methods for using the anti-ROR1 antibodies include in vitro and in vivo methods for binding ROR1, detecting ROR1 and treating diseases associated with ROR1 expression.

The present disclosure provides antigen binding proteins that bind specifically to a ROR1 polypeptide (e.g., antigen target) or fragment of the ROR1 polypeptide. In embodiments, the ROR1 target antigen comprises a naturally-occurring polypeptide (e.g., UniProt accession No. Q01973-1) having a wild-type or polymorphic or mutant amino acid sequence. The ROR1 target antigen can be prepared by recombinant methods or can be chemically synthesized. The ROR1 target antigen can be in soluble form or membrane-bound form (e.g., expressed by a cell or phage).

In embodiments, the ROR1 target antigen is expressed by a cell, for example a cancer or non-cancer cell line that naturally expresses ROR1 or is engineered to express ROR1, such as A549, U-2197, ASC TERT1, CACO-2, or HHSteC. Cell lines that do not express ROR1 are not expected to bind an anti-ROR1 antibody, such as for example Jurkat, Daudi, or K562 cell lines. The ROR1 target antigen can be a fusion protein or conjugated for example with a detectable moiety such as a fluorophore. The ROR1 target antigen can be a fusion protein or conjugated with an affinity tag, such as for example a His-tag. In embodiments, human ROR1 target antigen comprises the amino acid sequence of SEQ ID NO: 1 (e.g., UniProt accession No. Q01973-1) or SEQ ID NO:2 (e.g., recombinant his-tagged human ROR1 ECD from Acro Biosystems Cat. No. RO1-H522Y).

The present disclosure provides a fully human antibody of an IgG class that binds to a ROR1 polypeptide. In embodiments, the anti-ROR1 antibody comprises a heavy chain variable region having at least 95% sequence identity, or at least 96% sequence identity, or at least 97% sequence identity, or at least 98% sequence identity, or at least 99% sequence identity to the amino acid sequence of SEQ ID NO: 10, 20, 30 or 40, or combinations thereof; and/or the anti-ROR1 antibody comprises a light chain variable region having 95% sequence identity, or at least 96% sequence identity, or at least 97% sequence identity, or at least 98% sequence identity, or at least 99% sequence identity to the amino acid sequence of SEQ ID NO:11, 21, 31, 41 or 51, or combinations thereof. In embodiments, the anti-ROR1 antibody comprises an IgG1, IgG2, IgG3 or IgG4 class antibody. In embodiments, the anti-ROR1 antibody comprises an IgG1 or IgG4 class antibody. In embodiments, the anti-ROR1 antibody comprises an IgG1 class antibody.

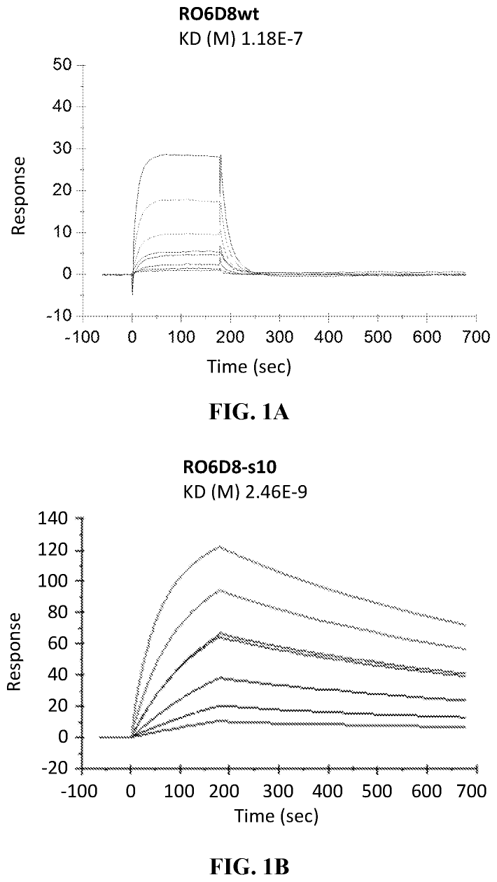

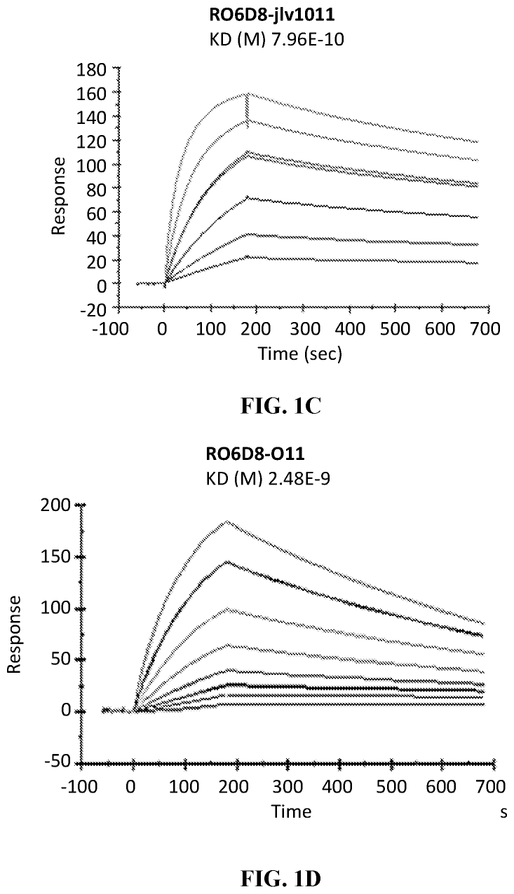

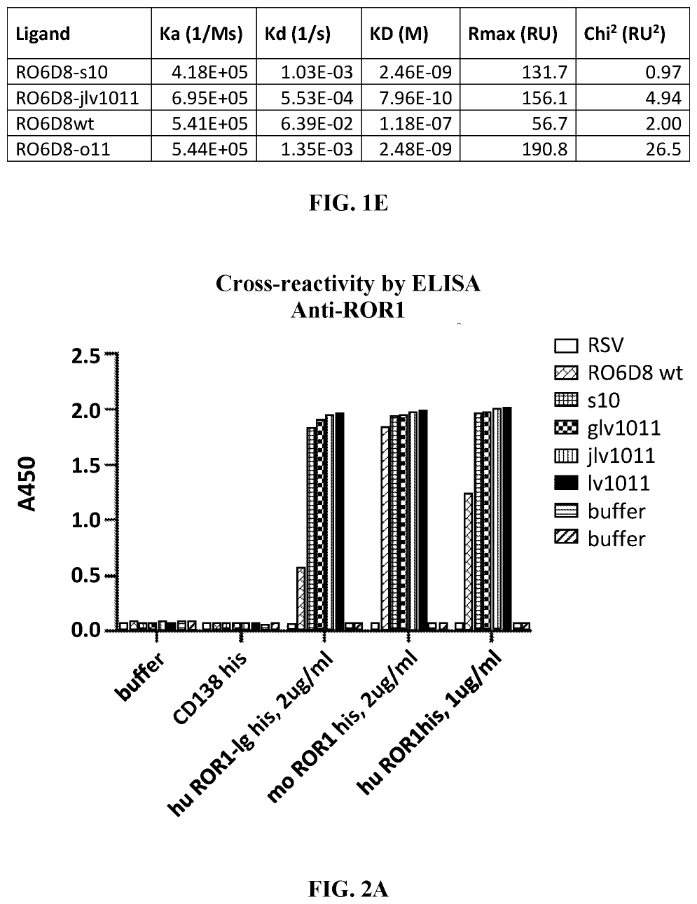

In embodiments, the anti-ROR1 antibody, or fragment thereof, comprises an antigen binding portion that binds an epitope of a ROR1 target antigen with a binding affinity (K_{D}) of 10⁻⁶ M or less, 10⁻⁷ M or less, 10⁻⁸ M or less, 10⁻⁹ M or less, or 10⁻¹⁰ M or less (see FIGS. 1A-E). In embodiments, the ROR1 antigen comprises a cell surface ROR1 antigen or a soluble ROR1 antigen. In embodiments, the ROR1 antigen comprises an extracellular portion of a cell surface ROR1 antigen. In embodiments, the ROR1 antigen comprises a human or non-human ROR1 antigen. In embodiments, the ROR1 antigen is expressed by a human or non-human cell. In embodiments, the anti-ROR1 antibody is expressed by many tissues during embryogenesis. In embodiments, the anti-ROR1 antibody is expressed by some B-cell malignancies, and various cancer cell lines. In embodiments, the anti-ROR1 antibody is expressed by some leukemias and lymphomas. In embodiments, the anti-ROR1 antibody binds a human ROR1 expressed by adenocarcinoma human alveolar basal epithelial cells (A549). In embodiments, the anti-ROR1 antibody binds a human ROR1 expressed by human chronic lymphocytic leukemia (CLL) B-cells. In embodiments, binding between the anti-ROR1 antibody, or fragment thereof, can be detected and measured using surface plasmon resonance, flow cytometry and/or ELISA.

The term “cross-reacts,” as used herein, refers to the ability of an antibody described herein to bind to ROR1 from a different species. The present disclosure provides an anti-ROR1 antibody which binds an epitope of ROR1 from a human, or can bind (e.g., cross-reactivity) with an epitope of ROR1 (e.g., homologous antigen) from any one or any combination of non-human animals such as mouse, rat, goat, rabbit, hamster and/or monkey (e.g., cynomolgus). In embodiments, the anti-ROR1 antibody or antigen-binding fragment binds human ROR1 (ECD) with a binding affinity K_{D} 10⁻⁵ M or less, or 10⁻⁶ M or less, or 10⁻⁷ M or less, or 10⁻⁸ M or less, or 10⁻⁹ M or less, or 10⁻¹⁰ M or less. In embodiments, the anti-ROR1 antibody or antigen-binding fragment binds human ROR1 Ig-like domain with a binding affinity K_{D} of 10⁻⁵ M or less, or 10⁻⁶ M or less, or 10⁻⁷ M or less, or 10⁻⁸ M or less, or 10⁻⁹ M or less, or 10⁻¹⁰ M or less. In embodiments, the anti-ROR1 antibody or antigen-binding fragment binds mouse ROR1 with a binding affinity K_{D} of 10⁻⁵ M or less, or 10⁻⁶ M or less, or 10⁻⁷ M or less, or 10⁻⁸ M or less, or 10⁻⁶ M or less, or 10⁻¹⁰ M or less.

In embodiments, human ROR1 (ECD) his is commercially available from Acro Biosystems (catalog #RO1-H522Y). In embodiments, human ROR1 Ig-like domain C-his is commercially available from Acro Biosystems (catalog #RO1-H5221). In embodiments, mouse ROR1 his is commercially available from Acro Biosystems (catalog #RO1-M5221).

The present disclosure provides a fully human antibody that binds ROR1 wherein the antibody comprising both heavy and light chains, wherein the heavy/light chain variable region amino acid sequences have at least 95% sequence identity, or at least 96% sequence identity, or at least 97% sequence identity, or at least 98% sequence identity, or at least 99% sequence identity to any of the following amino acid sequence sets: SEQ ID NOS: 10 and 11 (herein called RO6D8-s10), SEQ ID NOS: 20 and 21 (herein called RO6D8-jlv1011), SEQ ID NOS: 30 and 31 (herein called RO6D8-011), SEQ ID NOS: 40 and 41 (herein called RO6A-a7gm), or SEQ ID NOS: 40 and 51 (herein called RO6A-a8gm).

The present disclosure provides a Fab fully human antibody fragment, comprising a heavy variable region from a heavy chain and a variable region from a light chain, wherein the sequence of the variable region from the heavy chain is at least 95% identical, or at least 96% identical, or at least 97% identical, or at least 98% identical, or at least 99% identical to the amino acid sequence of SEQ ID NO: 10, 20, 30 or 40, or combinations thereof. The sequence of the variable region from the light chain is at least 95% identical, or at least 96% identical, or at least 97% identical, or at least 98% identical, or at least 99% identical to the amino acid sequence of SEQ ID NO:11, 21, 31, 41 or 51, or combinations thereof.

The present disclosure provides a Fab fully human antibody fragment, comprising a heavy chain variable region and a light chain variable region, wherein the heavy/light chain variable region amino acid sequences are at least 95% identical, or at least 96% identical, or at least 97% identical, or at least 98% identical, or at least 99% identical to any of the following amino acid sequence sets: SEQ ID NOS: SEQ ID NOS: 10 and 11 (herein called RO6D8-s10), SEQ ID NOS: 20 and 21 (herein called RO6D8-jlv1011), SEQ ID NOS: 30 and 31 (herein called RO6D8-011), SEQ ID NOS: 40 and 41 (herein called RO6A-a7gm), or SEQ ID NOS: 40 and 51 (herein called RO6A-a8gm).

The present disclosure provides a single chain fully human antibody comprising a polypeptide chain having a variable region from a fully human heavy chain and a variable region from a fully human light chain, and optionally a linker joining the variable heavy and variable light chain regions, wherein the variable heavy region comprises at least 95% sequence identity, or at least 96% sequence identity, or at least 97% sequence identity, or at least 98% sequence identity, or at least 99% sequence identity to the amino acid sequence of SEQ ID NO: 10, 20, 30 or 40, or combinations thereof. The variable light region comprises at least 95% sequence identity, or at least 96% sequence identity, or at least 97% sequence identity, or at least 98% sequence identity, or at least 99% sequence identity to the amino acid sequence of SEQ ID NO: 11, 21, 31, 41 or 51, or combinations thereof.

The present disclosure provides a single chain fully human antibody comprising a polypeptide chain having heavy chain variable region and a light chain variable region, wherein the heavy/light chain variable region amino acid sequence sets are at least 95% identical, or at least 96% identical, or at least 97% identical, or at least 98% identical, or at least 99% identical to any of the following amino acid sequence sets: SEQ ID NOS: SEQ ID NOS: 10 and 11 (herein called RO6D8-s10), SEQ ID NOS: 20 and 21 (herein called RO6D8-jlv1011), SEQ ID NOS: 30 and 31 (herein called RO6D8-011), SEQ ID NOS: 40 and 41 (herein called RO6A-a7gm), or SEQ ID NOS: 40 and 51 (herein called RO6A-a8gm).

The present disclosure provides pharmaceutical compositions comprising any of the anti-ROR1 antibodies described herein, or antigen binding protein thereof, in an admixture with a pharmaceutically-acceptable excipient. An excipient encompasses carriers and stabilizers. In embodiments, the pharmaceutical compositions comprise an anti-ROR1 antibody, or antigen binding fragment thereof, comprising a heavy chain variable region and a light chain variable region, wherein the heavy/light chain variable region amino acid sequences are at least 95% identical, or at least 96% identical, or at least 97% identical, or at least 98% identical, or at least 99% identical to any of the following amino acid sequence sets: SEQ ID NOS: SEQ ID NOS: SEQ ID NOS: 10 and 11 (herein called RO6D8-s10), SEQ ID NOS: 20 and 21 (herein called RO6D8-jlv1011), SEQ ID NOS: 30 and 31 (herein called RO6D8-011), SEQ ID NOS: 40 and 41 (herein called RO6A-a7gm), or SEQ ID NOS: 40 and 51 (herein called RO6A-a8gm).

The present disclosure provides a kit comprising any one or any combination of two or more of the anti-ROR1 antibodies, or antigen binding fragments thereof, described herein. In one embodiment, the kit comprises any one or any combination of two or more anti-ROR1 antibodies, or antigen binding fragments thereof, comprising a heavy chain variable region and a light chain variable region, wherein the heavy/light chain variable region amino acid sequences are at least 95% identical, or at least 96% identical, or at least 97% identical, or at least 98% identical, or at least 99% identical to any of the following amino acid sequence sets: SEQ ID NOS: SEQ ID NOS: SEQ ID NOS: 10 and 11 (herein called RO6D8-s10), SEQ ID NOS: 20 and 21 (herein called RO6D8-jlv1011), SEQ ID NOS: 30 and 31 (herein called RO6D8-011), SEQ ID NOS: 40 and 41 (herein called RO6A-a7gm), or SEQ ID NOS: 40 and 51 (herein called RO6A-a8gm).

The kit can be used to detect the presence or absence of a ROR1 antigen for example in a biological sample. The kit can be used for conducting an in vitro reaction such as antigen binding assays in the form of ELISA, flow cytometry or surface plasmon resonance; in vitro cell activation assays; luciferase-reporter assays; Western blotting and detection; and other such in vitro assays. The kit can be used for treating a subject having a ROR1-associated disease or condition, such as B-cell chronic lymphocytic leukemia (CLL).

The present disclosure provides a first nucleic acid encoding a first polypeptide comprising the anti-ROR1 antibody heavy chain variable region having at least 95% sequence identity, or at least 96% sequence identity, or at least 97% sequence identity, or at least 98% sequence identity, or at least 99% sequence identity with SEQ ID NO:10, 20, 30 or 40.

The present disclosure provides a first nucleic acid encoding a first polypeptide comprising the anti-ROR1 antibody (e.g., RO6D8-s10) heavy chain variable region having a heavy chain complementarity determining region 1 (CDR1) having the amino acid sequence of SEQ ID NO: 12, a heavy chain CDR2 region having the amino acid sequence of SEQ ID NO: 13, and a heavy chain CDR3 region having the amino acid sequence of SEQ ID NO:14.

The present disclosure provides a first nucleic acid encoding a first polypeptide comprising the anti-ROR1 antibody (e.g., RO6D8-jlv1011) heavy chain variable region having a heavy chain complementarity determining region 1 (CDR1) having the amino acid sequence of SEQ ID NO:22, a heavy chain CDR2 region having the amino acid sequence of SEQ ID NO:23, and a heavy chain CDR3 region having the amino acid sequence of SEQ ID NO:24.

The present disclosure provides a first nucleic acid encoding a first polypeptide comprising the anti-ROR1 antibody (e.g., RO6D8-011) heavy chain variable region having a heavy chain complementarity determining region 1 (CDR1) having the amino acid sequence of SEQ ID NO:32, a heavy chain CDR2 region having the amino acid sequence of SEQ ID NO:33, and a heavy chain CDR3 region having the amino acid sequence of SEQ ID NO:34.

The present disclosure provides a first nucleic acid encoding a first polypeptide comprising the anti-ROR1 antibody (e.g., RO6A-a7gm) heavy chain variable region having a heavy chain complementarity determining region 1 (CDR1) having the amino acid sequence of SEQ ID NO:42, a heavy chain CDR2 region having the amino acid sequence of SEQ ID NO:43, and a heavy chain CDR3 region having the amino acid sequence of SEQ ID NO:44.

The present disclosure provides a first nucleic acid encoding a first polypeptide comprising the anti-ROR1 antibody (e.g., RO6A-a8gm) heavy chain variable region having a heavy chain complementarity determining region 1 (CDR1) having the amino acid sequence of SEQ ID NO:42, a heavy chain CDR2 region having the amino acid sequence of SEQ ID NO:43, and a heavy chain CDR3 region having the amino acid sequence of SEQ ID NO:44.

The present disclosure provides a first vector operably linked to a first nucleic acid encoding a first polypeptide comprising the anti-ROR1 antibody heavy chain variable region having at least 95% sequence identity, or at least 96% sequence identity, or at least 97% sequence identity, or at least 98% sequence identity, or at least 99% sequence identity with SEQ ID NO: 10, 20, 30 or 40. In one embodiment, the first vector comprises an expression vector. In one embodiment, the first vector comprises at least one promoter which is operably linked to the first nucleic acid.

The present disclosure provides a first vector operably linked to a first nucleic acid encoding a first polypeptide comprising the anti-ROR1 antibody (e.g., RO6D8-s10) heavy chain variable region having a heavy chain complementarity determining region 1 (CDR1) having the amino acid sequence of SEQ ID NO:12, a heavy chain CDR2 region having the amino acid sequence of SEQ ID NO:13, and a heavy chain CDR3 region having the amino acid sequence of SEQ ID NO:14. In one embodiment, the first vector comprises a first expression vector. In one embodiment, the first vector comprises at least one promoter which is operably linked to the first nucleic acid.

The present disclosure provides a first vector operably linked to a first nucleic acid encoding a first polypeptide comprising the anti-ROR1 antibody (e.g., RO6D8-jlv1011) heavy chain variable region having a heavy chain complementarity determining region 1 (CDR1) having the amino acid sequence of SEQ ID NO:22, a heavy chain CDR2 region having the amino acid sequence of SEQ ID NO:23, and a heavy chain CDR3 region having the amino acid sequence of SEQ ID NO:24. In one embodiment, the first vector comprises a first expression vector. In one embodiment, the first vector comprises at least one promoter which is operably linked to the first nucleic acid.

The present disclosure provides a first vector operably linked to a first nucleic acid encoding a first polypeptide comprising the anti-ROR1 antibody (e.g., RO6D8-011) heavy chain variable region having a heavy chain complementarity determining region 1 (CDR1) having the amino acid sequence of SEQ ID NO:32, a heavy chain CDR2 region having the amino acid sequence of SEQ ID NO:33, and a heavy chain CDR3 region having the amino acid sequence of SEQ ID NO:34. In one embodiment, the first vector comprises a first expression vector. In one embodiment, the first vector comprises at least one promoter which is operably linked to the first nucleic acid.

The present disclosure provides a first vector operably linked to a first nucleic acid encoding a first polypeptide comprising the anti-ROR1 antibody (e.g., RO6A-a7gm) heavy chain variable region having a heavy chain complementarity determining region 1 (CDR1) having the amino acid sequence of SEQ ID NO:42, a heavy chain CDR2 region having the amino acid sequence of SEQ ID NO:43, and a heavy chain CDR3 region having the amino acid sequence of SEQ ID NO:44. In one embodiment, the first vector comprises a first expression vector. In one embodiment, the first vector comprises at least one promoter which is operably linked to the first nucleic acid.

The present disclosure provides a first vector operably linked to a first nucleic acid encoding a first polypeptide comprising the anti-ROR1 antibody (e.g., RO6A-a8gm) heavy chain variable region having a heavy chain complementarity determining region 1 (CDR1) having the amino acid sequence of SEQ ID NO:42, a heavy chain CDR2 region having the amino acid sequence of SEQ ID NO:43, and a heavy chain CDR3 region having the amino acid sequence of SEQ ID NO:44. In one embodiment, the first vector comprises a first expression vector. In one embodiment, the first vector comprises at least one promoter which is operably linked to the first nucleic acid.

The present disclosure provides a first host cell harboring the first vector operably linked to the first nucleic acid which encodes the anti-ROR1 antibody heavy chain variable region having at least 95% sequence identity, or at least 96% sequence identity, or at least 97% sequence identity, or at least 98% sequence identity, or at least 99% sequence identity with SEQ ID NO: 10, 20, 30 or 40. In one embodiment, the first vector comprises a first expression vector. In one embodiment, the first host cell expresses the first polypeptide comprising the antibody heavy chain variable region having at least 95% sequence identity to the amino acid sequence of SEQ ID NO: 10, 20, 30 or 40.

The present disclosure provides a method for preparing a first polypeptide having an antibody heavy chain variable region, the method comprising: culturing a population of the first host cells (e.g., a plurality of the first host cell) harboring the first expression vector under conditions suitable for expressing the first polypeptide having the antibody heavy chain variable region having at least 95% sequence identity to the amino acid sequence of SEQ ID NO:10, 20, 30 or 40. In one embodiment, the method further comprises: recovering from the population of the first host cells the expressed first polypeptide having at least 95% sequence identity to the amino acid sequence of SEQ ID NO:10, 20, 30 or 40.

The present disclosure provides a second nucleic acid encoding a second polypeptide comprising the anti-ROR1 antibody light chain variable region having at least 95% sequence identity, or at least 96% sequence identity, or at least 97% sequence identity, or at least 98% sequence identity, or at least 99% sequence identity with SEQ ID NO:11, 21, 31, 41 or 51.

The present disclosure provides a second nucleic acid encoding a second polypeptide comprising the anti-ROR1 antibody (e.g., RO6D8-s10) light chain variable region having a light chain complementarity determining region 1 (CDR1) having the amino acid sequence of SEQ ID NO: 15, a light chain CDR2 region having the amino acid sequence of SEQ ID NO:16, and a light chain CDR3 region having the amino acid sequence of SEQ ID NO:17.

The present disclosure provides a second nucleic acid encoding a second polypeptide comprising the anti-ROR1 antibody (e.g., RO6D8-jlv1011) light chain variable region having a light chain complementarity determining region 1 (CDR1) having the amino acid sequence of SEQ ID NO:25, a light chain CDR2 region having the amino acid sequence of SEQ ID NO:26, and a light chain CDR3 region having the amino acid sequence of SEQ ID NO:27.

The present disclosure provides a second nucleic acid encoding a second polypeptide comprising the anti-ROR1 antibody (e.g., RO6D8-011) light chain variable region having a light chain complementarity determining region 1 (CDR1) having the amino acid sequence of SEQ ID NO: 35, a light chain CDR2 region having the amino acid sequence of SEQ ID NO:36, and a light chain CDR3 region having the amino acid sequence of SEQ ID NO:37.

The present disclosure provides a second nucleic acid encoding a second polypeptide comprising the anti-ROR1 antibody (e.g., RO6A-a7gm) light chain variable region having a light chain complementarity determining region 1 (CDR1) having the amino acid sequence of SEQ ID NO: 45, a light chain CDR2 region having the amino acid sequence of SEQ ID NO:46, and a light chain CDR3 region having the amino acid sequence of SEQ ID NO:47.

The present disclosure provides a second nucleic acid encoding a second polypeptide comprising the anti-ROR1 antibody (e.g., RO6A-a8gm) light chain variable region having a light chain complementarity determining region 1 (CDR1) having the amino acid sequence of SEQ ID NO: 55, a light chain CDR2 region having the amino acid sequence of SEQ ID NO:56, and a light chain CDR3 region having the amino acid sequence of SEQ ID NO:57.

The present disclosure provides a second vector operably linked to a second nucleic acid encoding a second polypeptide comprising the anti-ROR1 antibody light chain variable region having at least 95% sequence identity, or at least 96% sequence identity, or at least 97% sequence identity, or at least 98% sequence identity, or at least 99% sequence identity with SEQ ID NO: 11, 21, 31, 41 or 51. In one embodiment, the second vector comprises a second expression vector. In one embodiment, the second vector comprises at least one promoter which is operably linked to the second nucleic acid.

The present disclosure provides a second vector operably linked to a second nucleic acid encoding a second polypeptide comprising the anti-ROR1 antibody (e.g., RO6D8-s10) light chain variable region having a light chain complementarity determining region 1 (CDR1) having the amino acid sequence of SEQ ID NO: 15, a light chain CDR2 region having the amino acid sequence of SEQ ID NO: 16, and a light chain CDR3 region having the amino acid sequence of SEQ ID NO:17. In one embodiment, the second vector comprises a second expression vector. In one embodiment, the second vector comprises at least one promoter which is operably linked to the second nucleic acid.

The present disclosure provides a second vector operably linked to a second nucleic acid encoding a second polypeptide comprising the anti-ROR1 antibody (e.g., RO6D8-jlv1011) light chain variable region having a light chain complementarity determining region 1 (CDR1) having the amino acid sequence of SEQ ID NO:25, a light chain CDR2 region having the amino acid sequence of SEQ ID NO:26, and a light chain CDR3 region having the amino acid sequence of SEQ ID NO:27. In one embodiment, the second vector comprises a second expression vector. In one embodiment, the second vector comprises at least one promoter which is operably linked to the second nucleic acid.

The present disclosure provides a second vector operably linked to a second nucleic acid encoding a second polypeptide comprising the anti-ROR1 antibody (e.g., RO6D8-011) light chain variable region having a light chain complementarity determining region 1 (CDR1) having the amino acid sequence of SEQ ID NO:35, a light chain CDR2 region having the amino acid sequence of SEQ ID NO:36, and a light chain CDR3 region having the amino acid sequence of SEQ ID NO:37. In one embodiment, the second vector comprises a second expression vector. In one embodiment, the second vector comprises at least one promoter which is operably linked to the second nucleic acid.

The present disclosure provides a second vector operably linked to a second nucleic acid encoding a second polypeptide comprising the anti-ROR1 antibody (e.g., RO6A-a7gm) light chain variable region having a light chain complementarity determining region 1 (CDR1) having the amino acid sequence of SEQ ID NO:45, a light chain CDR2 region having the amino acid sequence of SEQ ID NO:46, and a light chain CDR3 region having the amino acid sequence of SEQ ID NO:47. In one embodiment, the second vector comprises a second expression vector. In one embodiment, the second vector comprises at least one promoter which is operably linked to the second nucleic acid.

The present disclosure provides a second vector operably linked to a second nucleic acid encoding a second polypeptide comprising the anti-ROR1 antibody (e.g., RO6A-a8gm) light chain variable region having a light chain complementarity determining region 1 (CDR1) having the amino acid sequence of SEQ ID NO:55, a light chain CDR2 region having the amino acid sequence of SEQ ID NO:56, and a light chain CDR3 region having the amino acid sequence of SEQ ID NO:57. In one embodiment, the second vector comprises a second expression vector. In one embodiment, the second vector comprises at least one promoter which is operably linked to the second nucleic acid.

The present disclosure provides a second host cell harboring the second vector operably linked to the second nucleic acid which encodes the anti-ROR1 antibody light chain variable region having at least 95% sequence identity, or at least 96% sequence identity, or at least 97% sequence identity, or at least 98% sequence identity, or at least 99% sequence identity with SEQ ID NO:11, 21, 31, 41 or 51. In one embodiment, the second vector comprises a second expression vector. In one embodiment, the second host cell expresses the second polypeptide comprising the antibody light chain variable region having at least 95% sequence identity to the amino acid sequence of SEQ ID NO: 11, 21, 31, 41 or 51.

The present disclosure provides a method for preparing a second polypeptide having an antibody light chain variable region, the method comprising: culturing a population of the second host cells (e.g., a plurality of the second host cell) harboring the second expression vector under conditions suitable for expressing the second polypeptide having the antibody light chain variable region having at least 95% sequence identity to the amino acid sequence of SEQ ID NO: 11, 21, 31, 41 or 51. In one embodiment, the method further comprises: recovering from the population of the second host cells the expressed second polypeptide having at least 95% sequence identity to the amino acid sequence of SEQ ID NO: 11, 21, 31, 41 or 51.

The present disclosure provides a first and second nucleic acid, wherein (a) the first nucleic acid encodes a first polypeptide comprising the anti-ROR1 antibody heavy chain variable region having at least 95% sequence identity, or at least 96% sequence identity, or at least 97% sequence identity, or at least 98% sequence identity, or at least 99% sequence identity with SEQ ID NO: 10, 20, 30 or 40, and (b) the second polypeptide comprising the anti-ROR1 antibody light chain variable region having at least 95% sequence identity, or at least 96% sequence identity, or at least 97% sequence identity, or at least 98% sequence identity, or at least 99% sequence identity with SEQ ID NO:11, 21, 31, 41 or 51.

The present disclosure provides a vector operably linked to a first and a second nucleic acid, wherein (a) the first nucleic acid encodes a first polypeptide comprising the anti-ROR1 antibody heavy chain variable region having at least 95% sequence identity, or at least 96% sequence identity, or at least 97% sequence identity, or at least 98% sequence identity, or at least 99% sequence identity with SEQ ID NO:10, 20, 30 or 40, and (b) the second polypeptide comprising the anti-ROR1 antibody light chain variable region having at least 95% sequence identity, or at least 96% sequence identity, or at least 97% sequence identity, or at least 98% sequence identity, or at least 99% sequence identity with SEQ ID NO:11, 21, 31, 41 or 51. In one embodiment, the vector comprises an expression vector. In one embodiment, the vector comprises at least a first promoter which is operably linked to the first nucleic acid. In one embodiment, the vector comprises at least a second promoter which is operably linked to the second nucleic acid.

The present disclosure provides a host cell harboring a vector operably linked to a first and second nucleic acid, wherein (a) the first nucleic acid encodes a first polypeptide comprising the anti-ROR1 antibody heavy chain variable region having at least 95% sequence identity, or at least 96% sequence identity, or at least 97% sequence identity, or at least 98% sequence identity, or at least 99% sequence identity with SEQ ID NO: 10, 20, 30 or 40, and (b) the second nucleic acid encodes a second polypeptide comprising the anti-ROR1 antibody light chain variable region having at least 95% sequence identity, or at least 96% sequence identity, or at least 97% sequence identity, or at least 98% sequence identity, or at least 99% sequence identity with SEQ ID NO: 11, 21, 31, 41 or 51. In one embodiment, the vector comprises an expression vector. In one embodiment, the host cell expresses (a) the first polypeptide comprising the antibody heavy chain variable region having at least 95% sequence identity to the amino acid sequence of SEQ ID NO:10, 20, 30 or 40 and (b) the second polypeptide comprising the antibody light chain variable region having at least 95% sequence identity to the amino acid sequence of SEQ ID NO:11, 21, 31, 41 or 51.

The present disclosure provides a method for preparing a first polypeptide having an antibody heavy chain variable region and a second polypeptide having an antibody light chain variable region, the method comprising: culturing a population of the host cells (e.g., a plurality of the host cell) harboring an expression vector which is operably linked to a first and a second nucleic acid encoding the first and second polypeptides, respectively. In one embodiment, the culturing is conducted under conditions suitable for expressing (a) the first polypeptide having the antibody heavy chain variable region having at least 95% sequence identity to the amino acid sequence of SEQ ID NO:10, 20, 30 or 40, and (b) the second polypeptide having the antibody light chain variable region having at least 95% sequence identity to the amino acid sequence of SEQ ID NO:11, 21, 31, 41 or 51. In one embodiment, the method further comprises: recovering from the population of the host cells the expressed first polypeptide having the antibody heavy chain variable region having at least 95% sequence identity to the amino acid sequence of SEQ ID NO: 10, 20, 30 or 40 and the expressed second polypeptide having at least 95% sequence identity to the amino acid sequence of SEQ ID NO: 11, 21, 31, 41 or 51.

In one embodiment, the host cell, or population of host cells, harbor one or more expression vectors that can direct transient introduction of the transgene into the host cells or stable insertion of the transgene into the host cells' genome, where the transgene comprises nucleic acids encoding any of the first and/or second polypeptides described herein. The expression vector(s) can direct transcription and/or translation of the transgene in the host cell. The expression vectors can include one or more regulatory sequences, such as inducible and/or constitutive promoters and enhancers. The expression vectors can include ribosomal binding sites and/or polyadenylation sites. In one embodiment, the expression vector, which is operably linked to the nucleic acid encoding the first and/or second polypeptide, can direct production of the first and/or second polypeptide which can be displayed on the surface of the transgenic host cell, or the first and/or second polypeptide can be secreted into the cell culture medium.

The present disclosure provides methods for inhibiting growth or proliferation of target cells, or methods for killing target cells, the method comprising: contacting a population of effector cells with a population of target cells (e.g., target cells expressing ROR1) in the presence of an anti-ROR1 antibody (or antibody fragment thereof) under conditions that are suitable for killing the target cells. In embodiments, the population of effector cells comprises peripheral blood mononuclear cells (PBMCs) or natural killer (NK) cells. The PBMCs can include lymphocytes, including T cells, B cells and/or NK cells. In embodiments, the population of target cells comprise cells that naturally express ROR1, including mantle cell lymphoma (MCL), B-cell chronic lymphocytic leukemia (CLL) cells, or any type of solid tumor cells from a subject having a cancer associated with ROR1-expression. In embodiments, the population of target cells are any type of transgenic cells that are engineered to express ROR1. In embodiments, the ratio of effector to target cells can be about 1:1, or about 2:1, or about 3:1, or about 4:1, or about 5:1, or about 5-10:1, or about 10-20:1, or about 20-30:1.

The present disclosure provides methods for treating a subject having a disease associated with ROR1 expression, the method comprising: administering to the subject an effective amount of a therapeutic composition comprising an anti-ROR1 antibody or antigen binding fragment thereof, which is selected from a group consisting of any of the fully human anti-ROR1 antibodies described herein, any of the Fab fully human anti-ROR1 antibodies described herein, and any of the single chain human anti-ROR1 antibodies described herein. In embodiments, the disease associated with ROR1 expression is cancer. In embodiments, the disease associated with ROR1 expression comprises: chronic lymphocytic leukemia (CLL), breast cancer, lung cancer, gastric cancer, melanoma, colon cancer, renal cell carcinoma, and lymphomas.

In embodiments, the disease associated with ROR1 expression is cancer, including chronic lymphocytic leukemia (CLL), hairy cell leukemia (HCL), mantle cell lymphoma (MCL), diffuse large B-cell lymphoma (DLBCL), marginal zone lymphoma (MZL), follicular lymphoma (FL), chronic myeloid leukemia (CML), acute myeloid lymphoma (AML), myeloma, T-cell leukemia (TCL), Burkitt's lymphoma, multiple myeloma (MM), small lymphocytic lymphoma (SLL), non-Hodgkin lymphoma (NHL) that has undergone Richter's transformation, non-small cell lung cancer (NSCLC), hepatocellular carcinoma, pancreatic cancer, osteosarcoma, head and neck cancer, ovarian cancer, breast cancer, or triple negative breast cancer (TNBC). lymphoma, small lymphocytic lymphoma, marginal cell B-cell lymphoma, renal cell carcinoma, colon cancer, colorectal cancer, epithelial squamous cell cancer, melanoma, myeloma, stomach cancer, brain cancer, lung cancer, cervical cancer, liver cancer, bladder cancer, prostate cancer, testicular cancer, thyroid cancer.

In embodiments, the cancer is a metastatic cancer, refractory cancer, or recurrent cancer.

An anti-ROR1 antibody can be used alone to inhibit the growth of cancerous tumors. In embodiments, an anti-ROR1 antibody can be used in conjunction with another agent, e.g., other immunogenic agents, standard cancer treatments, or other antibodies, for treatment of a disease associated with ROR1 expression (or elevated ROR1 expression).

In embodiments, the disease associated with ROR1 expression is cancer. In embodiments, the method for treating a subject having a ROR1-expressing cancer, the method comprising: administering to the subject an effective amount of a therapeutic composition comprising an anti-ROR1 antibody or antigen binding fragment thereof, which is selected from a group consisting of any of the fully human anti-ROR1 antibodies described herein, any of the Fab fully human anti-ROR1 antibodies described herein, and any of the single chain human anti-ROR1 antibodies described herein. The method further comprising co-administration of a cytotoxic, cystostatic, or antiangiogenic agent suitable for treating the cancer. If the cancer is a B-cell malignancy, the method can further include, for example, co-administration of rituximab, alemtuzumab, ofatumumab, or a CHOP chemotherapeutic regimen.

LIST OF SEQUENCES:

Human ROR1 protein (UniProt Q01973-1) SEQ ID NO: 1:
MHRPRRRGTRPPLLALLAALLLAARGAAAQETELSVSAELVPTSSWNISSELNKDSYLTL
DEPMNNITTSLGQTAELHCKVSGNPPPTIRWFKNDAPVVQEPRRLSFRSTIYGSRLRIRN
LDTTDTGYFQCVATNGKEVVSSTGVLFVKFGPPPTASPGYSDEYEEDGFCQPYRGIACA
RFIGNRTVYMESLHMQGEIENQITAAFTMIGTSSHLSDKCSQFAIPSLCHYAFPYCDETSS
VPKPRDLCRDECEILENVLCQTEYIFARSNPMILMRLKLPNCEDLPQPESPEAANCIRIG
IPMADPINKNHKCYNSTGVDYRGTVSVTKSGRQCQPWNSQYPHTHTFTALRFPELNGG
HSYCRNPGNQKEAPWCFTLDENFKSDLCDIPACDSKDSKEKNKMEILYILVPSVAIPLAI
ALLFFFICVCRNNQKSSSAPVQRQPKHVRGQNVEMSMLNAYKPKSKAKELPLSAVRFM
EELGECAFGKIYKGHLYLPGMDHAQLVAIKTLKDYNNPQQWTEFQQEASLMAELHHPN
IVCLLGAVTQEQPVCMLFEYINQGDLHEFLIMRSPHSDVGCSSDEDGTVKSSLDHGDFL
HIAIQIAAGMEYLSSHFFVHKDLAARNILIGEQLHVKISDLGLSREIYSADYYRVQSKSLL
PIRWMPPEAIMYGKFSSDSDIWSFGVVLWEIFSFGLQPYYGFSNQEVIEMVRKRQLLPCS
EDCPPRMYSLMTECWNEIPSRRPRFKDIHVRLRSWEGLSSHTSSTTPSGGNATTQTTSLS
ASPVSNLSNPRYPNYMFPSQGITPQGQIAGFIGPPIPQNQRFIPINGYPIPPGYAAFPAAHY
QPTGPPRVIQHCPPPKSRSPSSASGSTSTGHVTSLPSSGSNQEANIPLLPHMSIPNHPGGMG
ITVFGNKSQKPYKIDSKQASLLGDANIHGHTESMISAEL.
Recombinant truncated human his-tag ROR1 extracellular domain protein (amino
acids 30-403 of SEQ ID NO: 1 with a carboxy terminal polyhistidine tag) SEQ
ID NO: 2:
QETELSVSAELVPTSSWNISSELNKDSYLTLDEPMNNITTSLGQTAELHCKVSGNPPPTIR
WFKNDAPVVQEPRRLSFRSTIYGSRLRIRNLDTTDTGYFQCVATNGKEVVSSTGVLFVK
FGPPPTASPGYSDEYEEDGFCQPYRGIACARFIGNRTVYMESLHMQGEIENQITAAFTMI
GTSSHLSDKCSQFAIPSLCHYAFPYCDETSSVPKPRDLCRDECEILENVLCQTEYIFARSN
PMILMRLKLPNCEDLPQPESPEAANCIRIGIPMADPINKNHKCYNSTGVDYRGTVSVTKS
GRQCQPWNSQYPHTHTFTALRFPELNGGHSYCRNPGNQKEAPWCFTLDENFKSDLCDIP
ACDSKDSKEKNKMEHHHHHH.
Recombinant truncated human his-tag ROR1 Ig-like domain (amino acids 39-151
of SEQ ID NO: 1 with a carboxy terminal polyhistidine tag) SEQ ID NO: 3:
ELVPTSSWNISSELNKDSYLTLDEPMNNITTSLGQTAELHCKVSGNPPPTIRWFKNDAPV
VQEPRRLSFRSTIYGSRLRIRNLDTTDTGYFQCVATNGKEVVSSTGVLFVKFGHHHHHH

TABLE 1

| Heavy chain variable domain: | Light chain variable domain: |
|---|---|
| RO6D8wt SEQ ID NO: 4 | RO6D8wt SEQ ID NO: 5 |
| QVQLVQSGAEVKKPGASVKVSCKASGY | AIQMTQSPSSLSASVGDRVTITCRASQDV |
| TFTNYYMHWVRQAPGQGLEWMGIINPS | RAHLAWYQQKPGKAPKLLIYAASSLQSG |
| GGSTSYAQKFQGRVTMTRDTSTSTVYM | VPSRFSGSGSGTDFTLTISSLQPEDFATYY |
| ELSSLRSEDTAVYYCARDSSSWYSGWY | CQQFNSYPITFGQGTRLEIK |
| FDLWGQGTTVTVSS | |
| RO6D8-s10 SEQ ID NO: 10 | RO6D8-s10 SEQ ID NO: 11 |
| QVQLVQSGAEVKKPGASVKVSCKASGY | AIQMTQSPSSLSASVGDRVTITCRASQGV |
| TFTNYYMHWVRQAPGQGLEWMGIINPS | STEIAWYQQKPGKAPKLLIYAASSLQSG |
| GGSTSYAQKFQGRVTMTRDTSTSTVYM | VPSRFSGSGSGTDFTLTISSLQPEDFATYY |
| ELSSLRSEDTAVYYCARSSRSSYYLWVL | CQQFNSYPITFGQGTRLEIK |
| DLWGQGTTVTVSS | |
| RO6D8-s10 SEQ ID NO: 12 | RO6D8-s10 SEQ ID NO: 15 |
| HC CDR1: NYYMH | LC CDR1: RASQGVSTEIA |
| RO6D8-s10 SEQ ID NO: 13 | RO6D8-s10 SEQ ID NO: 16 |
| HC CDR2: IINPSGGSTSYAQKFQG | LC CDR2: AASSLQS |
| RO6D8-s10 SEQ ID NO: 14 | RO6D8-s10 SEQ ID NO: 17 |
| HC CDR3: SSRSSYYLWVLDL | LC CDR3: QQFNSYPIT |
| RO6D8-jlv1011 SEQ ID NO: 20 | RO6D8-jlv1011 SEQ ID NO: 21 |
| QVQLVQSGAEVKKPGASVKVSCKASGY | AIQLTQSPSSLSASVGDRVTITCRASQGVS |
| TFTSKYYHWVRQAPGQGLEWMGIINPT | TEIAWYQQKPGKAPKLLIYAASSLQSGVP |
| SGSTSYAQKFQGRVTMTRDTSTSTVYM | SRFSGSGSGTDFTLTISSLQPEDFATYYCQ |
| ELSSLRSEDTAVYYCARDSSRYSGWYFD | QYYGYPIAFGQGTRLEIK |
| LWGQGTTVTVSS | |
| RO6D8-jlv1011 SEQ ID NO: 22 | RO6D8-jlv1011 SEQ ID NO: 25 |
| HC CDR1: SKYYH | LC CDR1: RASQGVSTEIA |
| RO6D8-jlv1011 SEQ ID NO: 23 | RO6D8-jlv1011 SEQ ID NO: 26 |
| HC CDR2: IINPTSGSTSYAQKFQG | LC CDR2: AASSLQS |
| RO6D8-jlv1011 SEQ ID NO: 24 | RO6D8-jlv1011 SEQ ID NO: 27 |
| HC CDR3: DSSRYSGWYFDL | LC CDR3: QQYYGYPIA |
| RO6D8-011 SEQ ID NO: 30 | RO6D8-011 SEQ ID NO: 31 |
| QVQLVQSGAEVKKPGASVKVSCKASGY | AIQMTQSPSSLSASVGDRVTITCRASQGIR |
| TFTNYYMHWVRQAPGQGLEWMGIINPT | TDLAWYQQKPGKAPKLLIYAASSLQSGV |
| SGRTSYAQKFQGRVTMTRDTSTSTVYM | PSRFSGSGSGTDFTLTISSLQPEDFATYYC |
| ELSSLRSEDTAVYYCARDSSSWYSGWY | QQYYGYPIAFGQGTRLEIK |
| FDLWGQGTTVTVSS | |
| RO6D8-011 SEQ ID NO: 32 | RO6D8-011 SEQ ID NO: 35 |
| HC CDR1: NYYMH | LC CDR1: RASQGIRTDLA |
| RO6D8-011 SEQ ID NO: 33 | RO6D8-011 SEQ ID NO: 36 |
| HC CDR2: IINPTSGRTSYAQKFQG | LC CDR2: AASSLQS |
| RO6D8-011 SEQ ID NO: 34 | RO6D8-011 SEQ ID NO: 37 |
| HC CDR3: DSSSWYSGWYFDL | LC CDR3: QQYYGYPIA |
| RO6A-a7gm SEQ ID NO: 40 | RO6A-a7gm SEQ ID NO: 41 |
| QVQLVESGGGLVKPGGSLRLSCAASGFT | QSALTQPASVSGSPGQSITISCTGTSSVSW |
| FSDYYMTWIRQAPGKGLEWVSYISGSSA | YQQHPGKAPKLMIYEVSKRPSGVSNRFS |
| YSNYADSVKGRFTISRDNSKNTLYLQM | GSKSGNTASLTISGLQAEDEADYYCSSYI |
| NSLRAEDTAVYYCARDPLLYGWLTDW | NDAVFFGGGTKLTVL |
| GQGTLVTVSS | |
| RO6A-a7gm SEQ ID NO: 42 | RO6A-a7gm SEQ ID NO: 45 |
| HC CDR1: DYYMT | LC CDR1: TGTSS |
| RO6A-a7gm SEQ ID NO: 43 | RO6A-a7gm SEQ ID NO: 46 |
| HC CDR2: YISGSSAYSNYADSVKG | LC CDR2: EVSKRPS |
| RO6A-a7gm SEQ ID NO: 44 | RO6A-a7gm SEQ ID NO: 47 |
| HC CDR3: DPLLYGWLTD | LC CDR3: SSYINDAVF |
| RO6A-a8gm SEQ ID NO: 40 | RO6A-a8gm SEQ ID NO: 51 |
| QVQLVESGGGLVKPGGSLRLSCAASGFT | QSALTQPASVSGSPGQSITISCTGTSSDGG |
| FSDYYMTWIRQAPGKGLEWVSYISGSSA | GYDSVSWYQQHPGKAPKLMIYDVNKRP |
| YSNYADSVKGRFTISRDNSKNTLYLQM | SGVSGRFSGSKSGNTASLTISGLQAEDEA |
| NSLRAEDTAVYYCARDPLLYGWLTDW | DYYCSSFTSDVMVFGGGTKLTVL |
| GQGTLVTVSS | |
| RO6A-a8gm SEQ ID NO: 42 | RO6A-a8gm SEQ ID NO: 55 |
| HC CDR1: DYYMT | LC CDR1: TGTSSDGGGYDSVS |
| RO6A-a8gm SEQ ID NO: 43 | RO6A-a8gm SEQ ID NO: 56 |
| HC CDR2: YISGSSAYSNYADSVKG | LC CDR2: DVNKRPS |
| RO6A-a8gm SEQ ID NO: 44 | RO6A-a8gm SEQ ID NO: 57 |
| HC CDR3: DPLLYGWLTD | LC CDR3: SSFTSDVMV |

### EXAMPLES

The following examples are meant to be illustrative and can be used to further understand embodiments of the present disclosure and should not be construed as limiting the scope of the present teachings in any way.

### Example 1: Measuring Binding Affinities Using Surface Plasmon Resonance

Binding kinetics of anti-ROR1 antibodies with his-tagged ROR1 protein were measured using surface plasmon resonance (SPR). The anti-ROR1 antibodies tested included proprietary antibodies RO6D8 wt, RO6D8-s10, RO6D8-jlv1011, and RO6D8-011. Anti-human fragment crystallizable region (Fc region) antibody was immobilized on a CM5 sensor chip to approximately 8,000 RU using standard N-hydroxysuccinimide/N-Ethyl-N′-(3-dimethylaminopropyl) carbodiimide hydrochloride (NHS/EDC) coupling methodology. The anti-ROR1 antibodies (1-2 μg/mL) were captured for 60 seconds at a flow rate of 10 μL/minute. The his-tagged ROR1 protein included amino acid 30 to amino acid 403 of SEQ ID NO: 1 (i.e., SEQ ID NO:2) (Acro Biosystems; Cat. No. RO1-H522Y). This polypeptide was serially diluted in a running buffer of 0.01 M HEPES pH 7.4, 0.15 M NaCl, 3 mM EDTA, 0.05% v/v Surfactant P20 (HBS-EP+) and run at 6 different dilutions. All measurements were conducted in HBS-EP+ buffer with a flow rate of 30 μL/minute. A 1:1 (Langmuir) binding model was used to fit the data. All BIACORE assays were performed at room temperature using Biacore T200 surface plasmon resonance (GE Healthcare).

The SPR sensorgrams of anti-ROR1 antibodies RO6D8 wt, RO6D8-s10, RO6D8-jlv1011, and RO6D8-O11 are shown in FIGS. 1A-1D, respectively, and their corresponding binding kinetics are listed in the table shown in FIG. 1E. Anti-human ROR1 antibodies RO6D8-s10, RO6D8-jlv1011, and RO6D8-011, showed an affinity in the nM range against their cognate antigen.

### Example 2: ELISA Cross-Reactivity

Cross-reactivity of the anti-human ROR1 antibodies with his-tagged ROR1 recombinant protein from human and mouse, were analyzed by ELISA assay. The antibodies 5 μg/mL (RO6D8wt, RO6D8-s10, RO6D8-glv1011, RO6D8-jlv1011, RO6D8-1v1011) were diluted with PBS buffer and coated on a plate at 50 μL per well or control buffers coated on a plate at 50 μL per well. The plate was washed three times with 150 μL/well of PBS-T (PBSIX supplemented with 0.05% Tween 20). Subsequently, the plate was blocked with 100 μL/well of blocking buffer (blocker Casein in PBS from Bioworld, Cat. No. 40320020-2) for 1 hour at RT. The plates were washed 3 times with 150 μL/well of PBS-T, after blocking. Recombinant mouse his-tag ROR1, 2 μg/ml (Acro Biosystems Cat. No. RO1-M5221, Lot: 1201-48ESI-65), human his-tag ROR1 extracellular domain (SEQ ID NO:2), 1 μg/ml (Acro Biosystems Cat. No. RO1-H522Y, lot: C81-76KF1-FR), human his-tag ROR1 Ig-like domain C-his, 2 μg/ml (Acro Biosystems Cat. No. RO1-H5221, Lot: B311-59SSI-CA), human his-tag CD138, 2 μg/ml (Sino Cat. No. 11429-H08H, Lot: LCL07NO0412) (negative control antibody), and Casein/PBS buffer (control). 30 μl of his-tagged ROR1 antigen, his tagged CD138 or Casein/PBS buffer were added to the plate, per well, and incubated for 1 hour at room temperature. The plate was washed three times with 150 μL/well of PBS-T and anti 6x-his (SEQ ID NO:58) tag antibody HRP (Abcam; Cat. No. Ab1187) was incubated at dilution 1:5,000, 30 μL/well for 1 hour at RT. Subsequently, the plate was washed three times with 150 μL/well of PBS-T and binding revealed by applying 30 μL/well of SureBlue™ TMB-1 component microwell peroxidase substrate (Thermo Scientific; Cat. No. 34028). The signal was stopped at the desired saturation point by using 15 μL/well of 2 N sulfuric acid (H₂SO₄) stop solution and read on a plate reader at 450 nm.

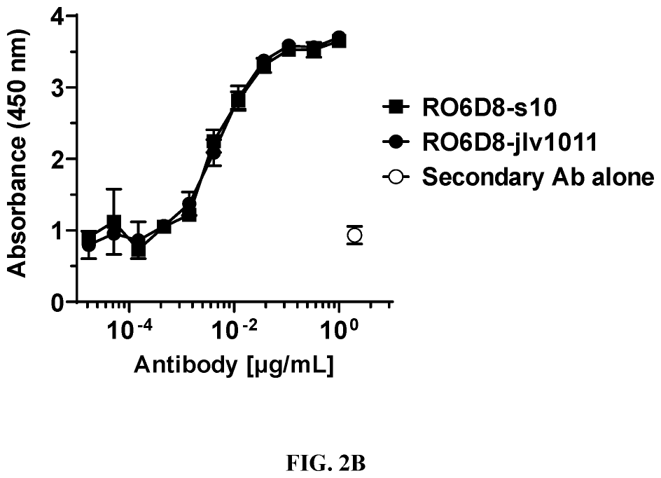

FIG. 2A shows that anti-human ROR1 antibodies RO6D8-s10, RO6D8-jlv1011, and RO6D8wt bind human ROR1 ECD (extracellular domain), human ROR1 Ig-like domain and mouse ROR1 protein.

In another experiment, cross-reactivity of the anti-human ROR1 antibodies at varying concentrations were analyzed using ELISA assay. On day 0: A 96-well plate (Corning; Cat #3690, lot #34117018) was coated with 50 μL/well of recombinant mouse ROR1-mouse IgG₂-Fc fusion protein at 2 μg/mL (R&D Systems; Cat #9910-RO-050, Lot No. DIWM0120121), the plate was sealed and incubated overnight at 4° C.

On day 1: The plate was washed with 150 μL/well of wash buffer (DPBS1X with 0.05% V/V Tween20). Non-specific binding was blocked by using blocking buffer (80 μL/well, DPBS1X with 2% BSA (Sigma Aldrich; Cat #AB412, Lot No. SLBT5979)+0.05% Tween20 (Sigma Aldrich; Cat #P9416-50 mL, Lot No. SLBW5532) and the plate was incubated at 37° C. for 1 hour. Next, the plate was washed twice with washing buffer. Human anti-ROR1 antibodies (RO6D8-jlv1011, RO6D8-s10) were incubated (80 μL/well) at concentrations ranging from 1.0E+00 to 1.7E-06 μg/mL (3-fold dilution) in blocking buffer. Two wells were not incubated with any anti-ROR1 antibody and were used for secondary antibody control only (negative control). The plate was incubated for 2 hours at RT on shaker. Then, the plate was washed thrice with washing buffer. Subsequently, a secondary goat anti-Human IgG-Fc, Mouse/Bovine/Horse SP ads-HRP (SouthernBiotech; Cat #2081-05; Lot No. L5311-TE40) was diluted at 1:2,000 in blocking buffer and 80 μL/well added to the wells. The plate was incubated for 1 hour at 37° C. in the dark. After three washings with wash buffer, 80 μL/well of SureBlue Reserve TMB 1-Component Microwell Peroxidase Substrate Solution (Sera Care; Cat #5120-0082) was added to each well and the plate was incubated 10-12 min at room temperature in the dark (plate was closely monitored and incubation was reduced or extended depending on color development). Color development was stopped by adding 50 μL/well of TMB Blue STOP Solution (Sera care; Cat #5150-0022) and the absorbance was read at 450 nm using a Tecan Spark.

FIG. 2B shows that anti-human ROR1 antibodies RO6D8-s10 and RO6D8-jlv1011 bind mouse ROR1 protein.

### Example 3: Cell Binding Assay by Flow Cytometry

Flow cytometry was used to test antibody binding to adenocarcinomic human alveolar basal epithelial cell line A549 (ROR1+) and immortalized line of human T lymphocyte cell line Jurkat (ROR1 negative), using various anti-ROR1 antibodies. The cells were prepared at concentration of 1×10⁶/ml in FACS buffer (PBS, 2% FBS, and 0.05% azide). Cells were plated at 30 μl/well in a V-bottom 96-well plate. Anti-ROR1 antibodies (with RO6D8wt used as control) were diluted in FACS buffer (5X serial dilution starting from 50 μg/ml) and 30 μl was added to each well with either A549 or Jurkat cells for 60 min on ice. After incubation, the cells were centrifuged at 2000 rpm for 5 minutes, flip out supernatant. After 1 wash with 200 μL/well of FACS buffer, cells were incubated with an AF647 goat anti-human Fab2 antibody (Jackson ImmunoResearch; Cat. No. 109-606-088) (dilution 1:2000 in FACS buffer) at 50 μL/well for 40 min on ice. After incubation, the cells were centrifuged at 2000 rpm for 5 minutes, flip out supernatant. After 1 wash with 200 μL/well of FACS buffer cells were resuspended in 30 μl of FACS buffer and acquired by flow cytometry using IntelliCyt readout.

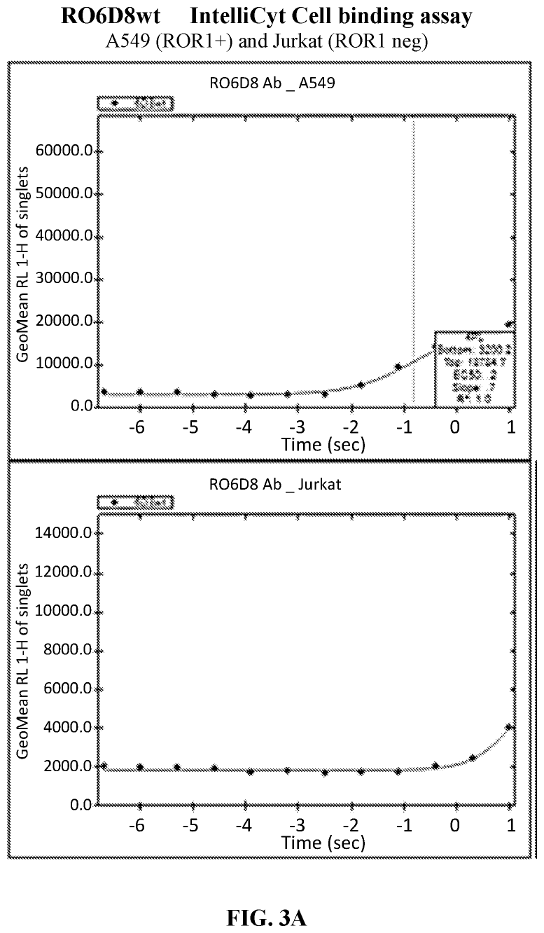

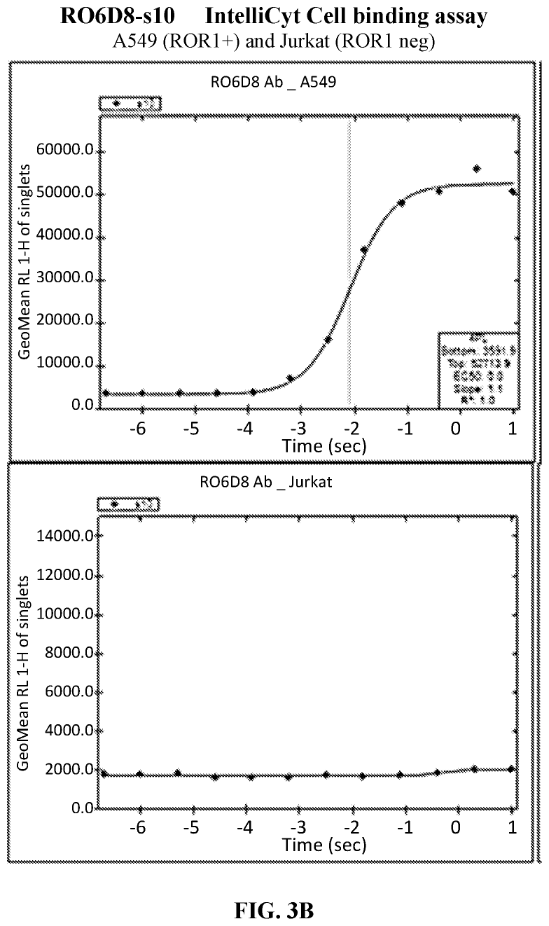

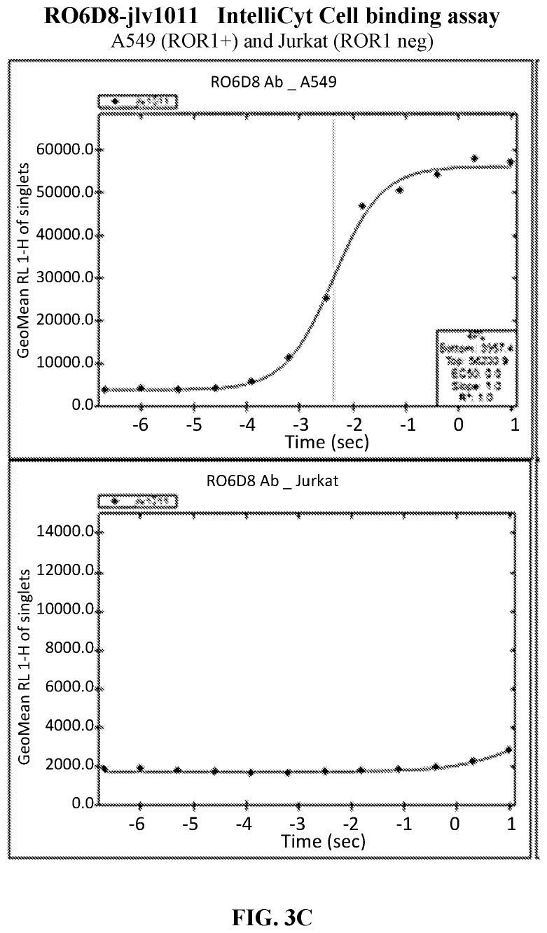

FIG. 3A shows wildtype anti-human ROR1 antibody binding to ROR1-expressing A549 cells and ROR1 negative Jurkat cells. FIGS. 3B and 3C show anti-human ROR1 antibodies RO6D8-s10 and RO6D8-jlv1011 binding to ROR1-expressing A549 cells and ROR1 negative Jurkat cells, respectively. Anti-human ROR1 antibodies RO6D8-s10 and RO6D8-jlv1011 showed dose-dependent binding to their cognate antigen expressed at the surface of a A549 cells, and no binding to ROR1 negative Jurkat cells. Anti-human ROR1 antibodies RO6D8-s10 and RO6D8-jlv1011, demonstrated stronger binding capacity, improved affinity and specificity as compared to the wildtype anti-human ROR1 antibody.

### Example 4: Cell Binding Assay by Flow Cytometry

Flow cytometry was used to test antibody binding to adenocarcinomic human alveolar basal epithelial cell line A549 (ROR1+), Burkitt's lymphoma cell line RAJI (ROR1+), breast cancer cell line (ROR1+), A549 ROR1-KO cell line (ROR1 knockout) which is ROR1 negative, and Duke's type B adenocarcinoma cell line LS174T (ROR1 negative).

30,000 cells were transferred into a V-bottom 96-well plate. The cells were spun down at 1,900 rpm for 3 min.

The cells were washed twice with cold FACS buffer (PBS1X+2% FCS+2 mM EDTA). The cells were spun down at 1,900 rpm for 2 minutes and supernatant was removed by quickly flicking the plate.

Another 96 well plate (round bottom, ultra low attachment, cat no #3474, Corning) was used for antibody dilution. All antibodies (RO6D8-s10, RO6D8-jlv1011, RO6A-a7gm, RO6A-a8gm, and isotype control IgG1) were 4-fold serially-diluted from a top conc of 10 μg/mL (10-0.0006 μg/mL) in FACS buffer (PBS+2% FCS+2 mM EDTA).

The cells were re-suspended in 100 μL/well of FACS buffer containing various concentrations of anti-ROR1 antibody and Isotype control IgG1 and incubated for 30 minutes at 4° C.

The cells were spun down at 1,900 rpm for 2 min and supernatant was removed the by quickly flicking the plate.

The cells were washed with 200 μL/well of FACS buffer. The cells were spun down at 1,900 rpm for 2 min and supernatant was removed by quickly flicking the plate. The washing step was repeated twice.

The cells were re-suspended in 120 L/well of FACS buffer containing Goat anti-human IgG AF647 (1:2,000, Southern Biotech, Cat No. 2040-31, Lot No. D1817-T817C) and plate was incubated for 20 minutes at 4° C. in the dark.

The cells were washed with 200 μL/well of FACS buffer. The cells were spun down at 1,900 rpm for 2 min and supernatant was removed by quickly flicking the plate. The washing step was repeated twice.

The cells were re-suspended in 120 μL/well of FACS buffer and 80 μL acquired by flow cytometry on the Attune NxT and data analyzed by using FlowJo.

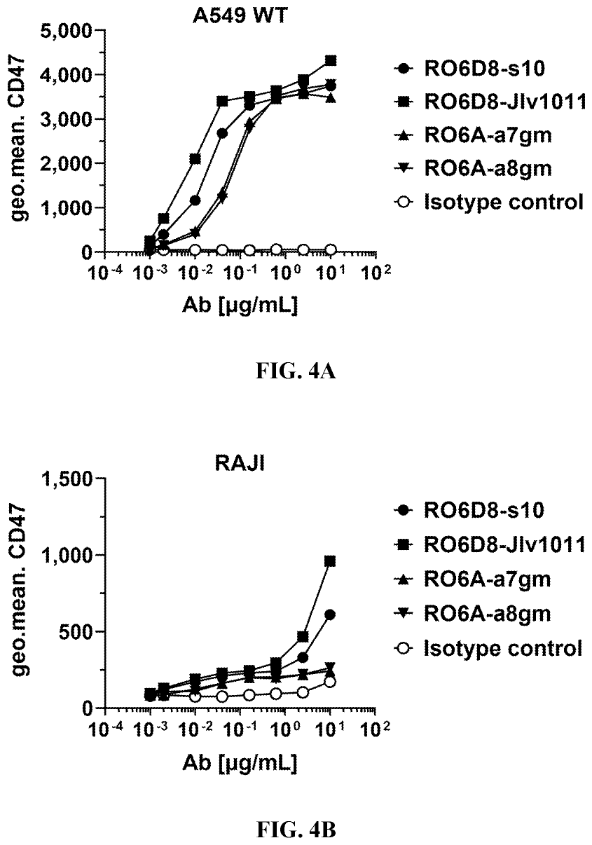

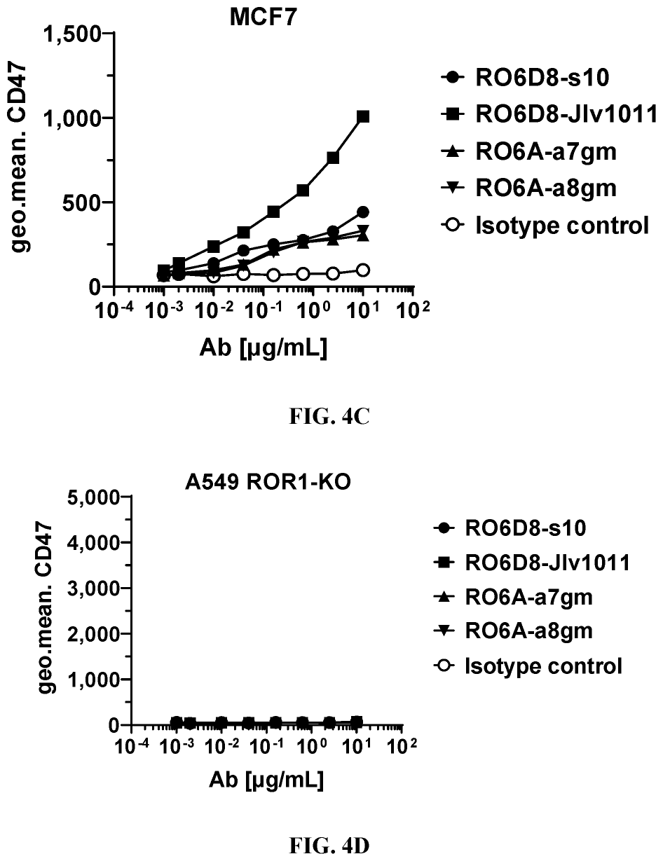

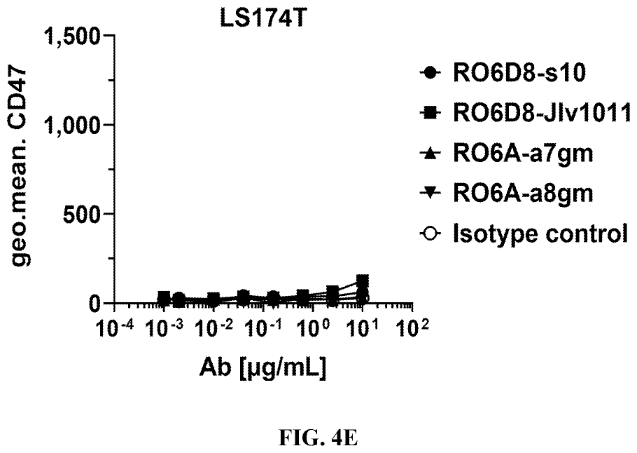

FIGS. 4A-E show anti-human ROR1 antibodies RO6D8-s10, RO6D8-jlv1011, RO6A-a7gm and RO6A-a8gm binding to ROR1-expressing A549 (FIG. 4A), Raji (FIG. 4B), and MCF7 (FIG. 4C) cells; and ROR1 negative A549 ROR1-KO (FIG. 4D) and LS174T (FIG. 4E) cells. Anti-human ROR1 antibodies RO6D8-s10, RO6D8-jlv1011, RO6A-a7gm and RO6A-a8gm showed dose-dependent binding to their cognate antigen expressed at the surface of a A549, Raji and MCF7 cells; and no binding to ROR1 negative A549 ROR1-KO and LS174T cells.

What is claimed:

1. An anti-ROR1 antigen-binding protein or fully human anti-ROR1 antibody, or an antigen-binding fragment thereof, comprising a heavy chain variable region and a light chain variable region,
wherein the heavy chain variable region comprises a heavy chain complementarity determining region 1 (CDR1) a heavy chain CDR2 and a heavy chain CDR3, and the light chain variable region comprises a light chain CDR1, a light chain CDR2, and a light chain CDR3; and
(a) the heavy chain CDR1 has the amino acid sequence of SEQ ID NO:12, the heavy chain CDR2 has the amino acid sequence of SEQ ID NO: 13, the heavy chain CDR3 has the amino acid sequence of SEQ ID NO: 14, the light chain CDR1 has the amino acid sequence of SEQ ID NO: 15, the light chain CDR2 has the amino acid sequence of SEQ ID NO: 16, and the light chain CDR3 has the amino acid sequence of SEQ ID NO: 17; (b) the heavy chain CDR1 has the amino acid sequence of SEQ ID NO:22, the heavy chain CDR2 has the amino acid sequence of SEQ ID NO:23, the heavy chain CDR3 has the amino acid sequence of SEQ ID NO:24, the light chain CDR1 has the amino acid sequence of SEQ ID NO:25, the light chain CDR2 has the amino acid sequence of SEQ ID NO: 26, and the light chain CDR3 has the amino acid sequence of SEQ ID NO:27; (c) the heavy chain CDR1 has the amino acid sequence of SEQ ID NO:32, the heavy chain CDR2 has the amino acid sequence of SEQ ID NO:33, the heavy chain CDR3 has the amino acid sequence of SEQ ID NO:34, the light chain CDR1 has the amino acid sequence of SEQ ID NO:35, the light chain CDR2 has the amino acid sequence of SEQ ID NO: 36, and the light chain CDR3 has the amino acid sequence of SEQ ID NO:37; (d) the heavy chain CDR1 has the amino acid sequence of SEQ ID NO:42, the heavy chain CDR2 has the amino acid sequence of SEQ ID NO:43, the heavy chain CDR3 has the amino acid sequence of SEQ ID NO:44, the light chain CDR1 has the amino acid sequence of SEQ ID NO:45, the light chain CDR2 has the amino acid sequence of SEQ ID NO: 46, and the light chain CDR3 has the amino acid sequence of SEQ ID NO:47; (e) the heavy chain CDR1 has the amino acid sequence of SEQ ID NO:42, the heavy chain CDR2 has the amino acid sequence of SEQ ID NO:43, the heavy chain CDR3 has the amino acid sequence of SEQ ID NO:44, the light chain CDR1 has the amino acid sequence of SEQ ID NO:55, the light chain CDR2 has the amino acid sequence of SEQ ID NO: 56, and the light chain CDR3 has the amino acid sequence of SEQ ID NO:57.

2. The antigen-binding protein, antibody or antigen-binding fragment thereof of claim 1, wherein the heavy chain variable region has at least 95% sequence identity to the amino acid sequence of SEQ ID NO: 10, 20, 30 or 40, and the light chain variable region has at least 95% sequence identity to the amino acid sequence of SEQ ID NO:11, 21, 31, 41 or 51.

3. An antigen-binding protein or fully human anti-ROR1 antibody, or an antigen-binding fragment thereof, comprising a heavy chain variable region and a light chain variable region, the heavy chain variable region having at least 95% sequence identity to the amino acid sequence of SEQ ID NO:10, 20, 30 or 40, and the light chain variable region having at least 95% sequence identity to the amino acid sequence of SEQ ID NO: 11, 21, 31, 41 or 51.

4. An antigen-binding protein or fully human anti-ROR1 antibody, or an antigen-binding fragment thereof, comprising a heavy chain variable region and a light chain variable region, wherein the heavy chain variable region and the light chain variable region comprise the amino acid sequences of SEQ ID NOS: 10 and 11, respectively (e.g., herein called RO6D8-s10), SEQ ID NOS: 20 and 21, respectively (e.g., herein called RO6D8-jlv1011), SEQ ID NOS: 30 and 31, respectively (e.g., herein called RO6D8-011), SEQ ID NOS: 40 and 41, respectively (e.g., herein called RO6A-a7gm), or SEQ ID NOS: 40 and 51, respectively (e.g., herein called RO6A-a8gm).

5. The antigen-binding fragment of any one of claims 1-4, comprising a Fab fragment.

6. The antigen-binding fragment of any one of claims 1-4, comprising a single chain antibody, wherein the heavy chain variable domain and the light chain variable domain are joined together with a peptide linker.

7. The antigen-binding protein, antibody or antigen-binding fragment thereof, of any one of the preceding claims, comprising an IgG antibody, which is IgG1, IgG2, IgG3 or IgG4 class antibody.

8. The antigen-binding protein, antibody or antigen-binding fragment thereof, of claim 7, comprising the IgG1 or IgG4 class antibody.

9. The antigen-binding protein, antibody or antigen-binding fragment thereof, of claim 7, comprising the IgG1 class antibody.

10. The antigen-binding protein, antibody or antigen-binding fragment thereof, of any one of the preceding claims, wherein the antigen-binding protein, antibody, or the antigen-binding fragment thereof, binds human ROR1 protein with a K_{D} of 10⁻⁷ M or less.

11. A pharmaceutical composition, comprising the antigen-binding protein, antibody or antigen-binding fragment of any one of the preceding claims and a pharmaceutically acceptable excipient.

12. A kit comprising the comprising the antigen-binding protein, antibody or antigen-binding fragment of any one of the preceding claims and a pharmaceutically acceptable excipient of any one of claims 1-10.

13. A nucleic acid that encodes the heavy chain variable region of the antigen-binding protein, antibody or antigen-binding fragment of any one of claims 1-10.

14. A nucleic acid that encodes the light chain variable region of the antigen-binding protein, antibody or antigen-binding fragment of any one of claims 1-10.

15. A nucleic acid that encodes (i) the heavy chain variable region of the antigen-binding protein, antibody or antigen-binding fragment of any one of claims 1-10, and (ii) the light chain variable region of the antigen-binding protein, antibody or antigen-binding fragment.

16. A vector comprising the nucleic acid of claim 13.

17. A vector comprising the nucleic acid of claim 14.

18. A vector comprising the nucleic acid of claim 15.

19. A host cell harboring the vector of claim 16.

20. The host cell of claim 19, wherein the vector comprises an expression vector, and wherein the host cell expresses the heavy chain variable region.

21. A host cell harboring the vector of claim 17.

22. The host cell of claim 21, wherein the vector comprises an expression vector, and wherein the host cell expresses the light chain variable region.

23. A host cell harboring a first vector comprising the vector of claim 16 and a second vector comprising the vector of claim 17.

24. The host cell of claim 23, wherein the first vector comprises a first expression vector, wherein the second vector comprises a second expression vector, and wherein the host cell expresses the heavy and the light chain variable regions.

25. A host cell harboring the vector of claim 18.

26. The host cell of claim 25, wherein the vector comprises an expression vector, and wherein the host cell expresses the heavy and the light chain variable regions.

27. A method for preparing a heavy chain variable region of an antigen-binding protein, antibody or antigen-binding fragment, the method comprising: culturing a population of the host cell of claim 20 under conditions suitable for expressing the heavy chain variable region of the antigen-binding protein, antibody or antigen-binding fragment.

28. The method of claim 27, further comprising: recovering from the host cells the expressed heavy chain variable region of the antigen-binding protein, antibody or antigen-binding fragment.

29. A method for preparing a light chain variable region of an antigen-binding protein, antibody or antigen-binding fragment, the method comprising: culturing a population of the host cell of claim 22 under conditions suitable for expressing the light chain variable region of the antigen-binding protein, antibody or antigen-binding fragment.

30. The method of claim 29, further comprising: recovering from the host cells the expressed light chain variable region of the antigen-binding protein, antibody or antigen-binding fragment.

31. A method for preparing (i) a heavy chain variable region of a antigen-binding protein, antibody or antigen-binding fragment, and (ii) a light chain variable region of an antigen-binding protein, antibody or antigen-binding fragment, the method comprising: culturing a population of the host cell of claim 24 under conditions suitable for expressing (i) the heavy chain variable region of the antigen-binding protein, antibody or antigen-binding fragment, and (ii) the light chain variable region of the antigen-binding protein, antibody or antigen-binding fragment.

32. The method of claim 31, further comprising: recovering from the host cells (i) the expressed heavy chain variable region of the antigen-binding protein, antibody or antigen-binding fragment, and (ii) the expressed light chain variable region of the antigen-binding protein, antibody or antigen-binding fragment.

33. A method for preparing (i) a heavy chain variable region of an antigen-binding protein, antibody or antigen-binding fragment, and (ii) a light chain variable region of a antigen-binding protein, antibody or antigen-binding fragment, the method comprising: culturing a population of the host cell of claim 26 under conditions suitable for expressing (i) the heavy chain variable region of the antigen-binding protein, antibody or antigen-binding fragment, and (ii) the light chain variable region of the antigen-binding protein, antibody or antigen-binding fragment.

34. The method of claim 33, further comprising: recovering from the host cells (i) the expressed heavy chain variable region of the antigen-binding protein, antibody or antigen-binding fragment, and (ii) the expressed light chain variable region of the antigen-binding protein, antibody or antigen-binding fragment.

35. A method for inhibiting growth or proliferation of ROR1-expressing cells, comprising: contacting (i) a population of effector cells with (ii) a population of target cells which express ROR1 (iii) in the presence of the human anti-ROR1 antibody of any one of claims 1-10, under conditions that are suitable for inhibiting growth or proliferation of the ROR1-expressing cells.

36. The method of claim 35, wherein the population of effector cells comprises PBMCs or NK cells.

37. The method of claim 35 or 36, wherein the population of target cells comprise ROR1 expressing human cancer cells or transgenic cells expressing ROR1.

38. The method of any one of claims 35-37, wherein the ratio of the effector-to-target cells is 1:1, 2:1, 3:1, 4:1 or 5:1.

39. The method of any one of claims 35-37, wherein the ratio of the effector-to-target cells is 5-10:1, 10-20:1, or 20-30:1.

40. A method for killing ROR1-expressing cells, comprising: contacting (i) a population of effector cells with (ii) a population of target cells which express ROR1 (iii) in the presence of the human anti-ROR1 antibody of any one of claims 1-10, under conditions that are suitable for inhibiting growth or proliferation of the ROR1-expressing cells.

41. The method of claim 40, wherein the population of effector cells comprises PBMCs or NK cells.

42. The method of claim 40 or 41, wherein the population of target cells comprise ROR1 expressing human cancer cells or transgenic cells expressing ROR1.

43. The method of any one of claims 40-42, wherein the ratio of the effector-to-target cells is 1:1, 2:1, 3:1, 4:1 or 5:1.

44. The method of any one of claims 40-42, wherein the ratio of the effector-to-target cells is 5-10:1, 10-20:1, or 20-30:1.

45. A method for treating a subject having a disease associated with ROR1 expression, the method comprising: administering to the subject an effective amount of a therapeutic composition comprising the antigen-binding protein, antibody or antigen-binding fragment of any one of claims 1-10.

46. The method of claim 45, wherein the disease associated with ROR1 expression is cancer.

47. The method of claim 46, wherein cancer is chronic lymphocytic leukemia (CLL), breast cancer, lung cancer, gastric cancer, melanoma, colon cancer, renal cell carcinoma, or lymphomas.

48. The method of claim 46, wherein cancer is chronic lymphocytic leukemia (CLL), hairy cell leukemia (HCL), mantle cell lymphoma (MCL), diffuse large B-cell lymphoma (DLBCL), marginal zone lymphoma (MZL), follicular lymphoma (FL), chronic myeloid leukemia (CML), acute myeloid lymphoma (AML), myeloma, T-cell leukemia (TCL), Burkitt's lymphoma, multiple myeloma (MM), small lymphocytic lymphoma (SLL), non-Hodgkin lymphoma (NHL) that has undergone Richter's transformation, non-small cell lung cancer (NSCLC), hepatocellular carcinoma, pancreatic cancer, osteosarcoma, head and neck cancer, ovarian cancer, breast cancer, or triple negative breast cancer (TNBC). lymphoma, small lymphocytic lymphoma, marginal cell B-cell lymphoma, renal cell carcinoma, colon cancer, colorectal cancer, epithelial squamous cell cancer, melanoma, myeloma, stomach cancer, brain cancer, lung cancer, cervical cancer, liver cancer, bladder cancer, prostate cancer, testicular cancer, or thyroid cancer.

49. The method of claim 46, wherein the cancer is a metastatic cancer, refractory cancer, or recurrent cancer.

50. The antigen-binding protein, antibody or antigen-binding fragment of any one of claims 1-10, for use in the method of any one of claims 27-49.


SEQUENCE LISTING (58 sequences)

SEQ ID NO: 1 - protein, 937 aa, Homo sapiens
MHRPRRRGTRPPLLALLAALLLAARGAAAQETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYILVPSVAIPLAIALLFFFICVCRNNQKSSSAPVQRQPKHVRGQNVEMSMLNAYKPKSKAKELPLSAVRFMEELGECAFGKIYKGHLYLPGMDHAQLVAIKTLKDYNNPQQWTEFQQEASLMAELHHPNIVCLLGAVTQEQPVCMLFEYINQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYMFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYKIDSKQASLLGDANIHGHTESMISAEL

SEQ ID NO: 2 - protein, 380 aa, Artificial Sequence
  Recombinant truncated human his-tag ROR1 extracellular domain protein (amino acids 30-403 of SEQ ID NO:1 with a carboxy terminal polyhistidine tag
QETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEHHHHHH

SEQ ID NO: 3 - protein, 119 aa, Artificial Sequence
  Recombinant truncated human his-tag ROR1 Ig-like domain (amino acids 39-151 of SEQ ID NO:1 with a carboxy terminal polyhistidine tag)
ELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGHHHHHH

SEQ ID NO: 4 - protein, 122 aa, Artificial Sequence
  RO6D8wt heavy chain variable domain
QVQLVQSGAEVKKPGASVKVSCKASGYTFTNYYMHWVRQAPGQGLEWMGIINPSGGSTSYAQKFQGRVTMTRDTSTSTVYMELSSLRSEDTAVYYCARDSSSWYSGWYFDLWGQGTTVTVSS

SEQ ID NO: 5 - protein, 107 aa, Artificial Sequence
  RO6D8wt light chain variable domain
AIQMTQSPSSLSASVGDRVTITCRASQDVRAHLAWYQQKPGKAPKLLIYAASSLQSGVPSRFSGSGSGTDFTLTISSLQPEDFATYYCQQFNSYPITFGQGTRLEIK

SEQ ID NO: 6-9 - skipped (4 entries)

SEQ ID NO: 10 - protein, 122 aa, Artificial Sequence
  RO6D8-s10 heavy chain variable domain
QVQLVQSGAEVKKPGASVKVSCKASGYTFTNYYMHWVRQAPGQGLEWMGIINPSGGSTSYAQKFQGRVTMTRDTSTSTVYMELSSLRSEDTAVYYCARSSRSSYYLWVLDLWGQGTTVTVSS

SEQ ID NO: 11 - protein, 107 aa, Artificial Sequence
  RO6D8-s10 light chain variable domain
AIQMTQSPSSLSASVGDRVTITCRASQGVSTEIAWYQQKPGKAPKLLIYAASSLQSGVPSRFSGSGSGTDFTLTISSLQPEDFATYYCQQFNSYPITFGQGTRLEIK

SEQ ID NO: 12 - protein, 5 aa, Artificial Sequence
  RO6D8-s10 HC CDR1
NYYMH

SEQ ID NO: 13 - protein, 17 aa, Artificial Sequence
  RO6D8-s10 HC CDR2
IINPSGGSTSYAQKFQG

SEQ ID NO: 14 - protein, 13 aa, Artificial Sequence
  RO6D8-s10 HC CDR3
SSRSSYYLWVLDL

SEQ ID NO: 15 - protein, 11 aa, Artificial Sequence
  RO6D8-s10 LC CDR1
RASQGVSTEIA

SEQ ID NO: 16 - protein, 7 aa, Artificial Sequence
  RO6D8-s10 LC CDR2
AASSLQS

SEQ ID NO: 17 - protein, 9 aa, Artificial Sequence
  RO6D8-s10 LC CDR3
QQFNSYPIT

SEQ ID NO: 18-19 - skipped (2 entries)

SEQ ID NO: 20 - protein, 121 aa, Artificial Sequence
  RO6D8-jlv1011 heavy chain variable domain
QVQLVQSGAEVKKPGASVKVSCKASGYTFTSKYYHWVRQAPGQGLEWMGIINPTSGSTSYAQKFQGRVTMTRDTSTSTVYMELSSLRSEDTAVYYCARDSSRYSGWYFDLWGQGTTVTVSS

SEQ ID NO: 21 - protein, 107 aa, Artificial Sequence
  RO6D8-jlv1011 light chain variable domain
AIQLTQSPSSLSASVGDRVTITCRASQGVSTEIAWYQQKPGKAPKLLIYAASSLQSGVPSRFSGSGSGTDFTLTISSLQPEDFATYYCQQYYGYPIAFGQGTRLEIK

SEQ ID NO: 22 - protein, 5 aa, Artificial Sequence
  RO6D8-jlv1011 HC CDR1
SKYYH

SEQ ID NO: 23 - protein, 17 aa, Artificial Sequence
  RO6D8-jlv1011 HC CDR2
IINPTSGSTSYAQKFQG

SEQ ID NO: 24 - protein, 12 aa, Artificial Sequence
  RO6D8-jlv1011 HC CDR3
DSSRYSGWYFDL

SEQ ID NO: 25 - protein, 11 aa, Artificial Sequence
  RO6D8-jlv1011 LC CDR1
RASQGVSTEIA

SEQ ID NO: 26 - protein, 7 aa, Artificial Sequence
  RO6D8-jlv1011 LC CDR2
AASSLQS

SEQ ID NO: 27 - protein, 9 aa, Artificial Sequence
  RO6D8-jlv1011 LC CDR3
QQYYGYPIA

SEQ ID NO: 28-29 - skipped (2 entries)

SEQ ID NO: 30 - protein, 122 aa, Artificial Sequence
  RO6D8-O11 heavy chain variable domain
QVQLVQSGAEVKKPGASVKVSCKASGYTFTNYYMHWVRQAPGQGLEWMGIINPTSGRTSYAQKFQGRVTMTRDTSTSTVYMELSSLRSEDTAVYYCARDSSSWYSGWYFDLWGQGTTVTVSS

SEQ ID NO: 31 - protein, 107 aa, Artificial Sequence
  RO6D8-O11 light chain variable domain
AIQMTQSPSSLSASVGDRVTITCRASQGIRTDLAWYQQKPGKAPKLLIYAASSLQSGVPSRFSGSGSGTDFTLTISSLQPEDFATYYCQQYYGYPIAFGQGTRLEIK

SEQ ID NO: 32 - protein, 5 aa, Artificial Sequence
  RO6D8-O11 HC CDR1
NYYMH

SEQ ID NO: 33 - protein, 17 aa, Artificial Sequence
  RO6D8-O11 HC CDR2
IINPTSGRTSYAQKFQG

SEQ ID NO: 34 - protein, 13 aa, Artificial Sequence
  RO6D8-O11 HC CDR3
DSSSWYSGWYFDL

SEQ ID NO: 35 - protein, 11 aa, Artificial Sequence
  RO6D8-O11 LC CDR1
RASQGIRTDLA

SEQ ID NO: 36 - protein, 7 aa, Artificial Sequence
  RO6D8-O11 LC CDR2
AASSLQS

SEQ ID NO: 37 - protein, 9 aa, Artificial Sequence
  RO6D8-O11 LC CDR3
QQYYGYPIA

SEQ ID NO: 38-39 - skipped (2 entries)

SEQ ID NO: 40 - protein, 119 aa, Artificial Sequence
  RO6A-a7gm / RO6A-a8gm heavy chain variable domain
QVQLVESGGGLVKPGGSLRLSCAASGFTFSDYYMTWIRQAPGKGLEWVSYISGSSAYSNYADSVKGRFTISRDNSKNTLYLQMNSLRAEDTAVYYCARDPLLYGWLTDWGQGTLVTVSS

SEQ ID NO: 41 - protein, 102 aa, Artificial Sequence
  RO6A-a7gm light chain variable domain
QSALTQPASVSGSPGQSITISCTGTSSVSWYQQHPGKAPKLMIYEVSKRPSGVSNRFSGSKSGNTASLTISGLQAEDEADYYCSSYINDAVFFGGGTKLTVL

SEQ ID NO: 42 - protein, 5 aa, Artificial Sequence
  RO6A-a7gm / RO6A-a8gm HC CDR1
DYYMT

SEQ ID NO: 43 - protein, 17 aa, Artificial Sequence
  RO6A-a7gm / RO6A-a8gm HC CDR2
YISGSSAYSNYADSVKG

SEQ ID NO: 44 - protein, 10 aa, Artificial Sequence
  RO6A-a7gm / RO6A-a8gm HC CDR3
DPLLYGWLTD

SEQ ID NO: 45 - protein, 5 aa, Artificial Sequence
  RO6A-a7gm LC CDR1
TGTSS

SEQ ID NO: 46 - protein, 7 aa, Artificial Sequence
  RO6A-a7gm LC CDR2
EVSKRPS

SEQ ID NO: 47 - protein, 9 aa, Artificial Sequence
  RO6A-a7gm LC CDR3
SSYINDAVF

SEQ ID NO: 48-50 - skipped (3 entries)

SEQ ID NO: 51 - protein, 109 aa, Artificial Sequence
  RO6A-a8gm light chain variable domain
QSALTQPASVSGSPGQSITISCTGTSSDGGGYDSVSWYQQHPGKAPKLMIYDVNKRPSGVSGRFSGSKSGNTASLTISGLQAEDEADYYCSSFTSDVMVFGGGTKLTVL

SEQ ID NO: 52-54 - skipped (3 entries)

SEQ ID NO: 55 - protein, 14 aa, Artificial Sequence
  RO6A-a8gm LC CDR1
TGTSSDGGGYDSVS

SEQ ID NO: 56 - protein, 7 aa, Artificial Sequence
  RO6A-a8gm LC CDR2
DVNKRPS

SEQ ID NO: 57 - protein, 9 aa, Artificial Sequence
  RO6A-a8gm LC CDR3
SSFTSDVMV

SEQ ID NO: 58 - protein, 6 aa, Artificial Sequence
  6x His tag
HHHHHH